(19) United States
(12) Patent Application Publication
(10) Pub. No.: US 2009/0192505 A1
(43) Pub. Date: Jul. 30, 2009

(54) Method for cryospray ablation

(75) Inventors: Timothy E. Askew, Baltimore, MD (US); William S. Krimsky, Bel Air, MD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Reset Medical, Inc., Baltimore, MD (US)

(21) Appl. No.: 12/314,245

(22) Filed: Dec. 5, 2008

Related U.S. Application Data
(60) Provisional application No. 60/992,586, filed on Dec. 5, 2007.
(60) Provisional application No. 61/100,216, filed on Sep. 25, 2008.

(51) Int. Cl. A61B 18/02; A61K 49/00; A61K 51/00; A61K 49/06; A61K 49/04; A61K 49/22

(52) U.S. Cl. 606/21; 424/9.1; 424/1.11; 424/9.3; 424/9.4; 424/9.5

(57) ABSTRACT

The present invention relates to methods for treating tissue in the thoracic cavity of a subject by the application of a cryogen, or using the cryogen to create an isotherm in proximity to the tissue to be treated. A wide variety of conditions may be treated using the methods of the invention including asthma, neoplastic disease and a variety of conditions characterized by inflammation in lung and chest tissue.

### FIELD OF THE INVENTION

The invention relates to methods of cryospray therapy and drug delivery for airway and thoracic applications.

### BACKGROUND

Over five million people in the United States suffer from acute and chronic benign pulmonary disease including, but are not limited to, asthma, bronchitis, and emphysema. This number is vastly greater for areas outside of the United States. For the majority of these people there is no cure and few effective treatment alternatives.

According to the American Lung Association, there were approximately 20 million Americans with asthma in 2002, 14 million of whom were adults. Asthma resulted in approximately 1.9 million emergency room visits in 2002, of which approximately 484,000 resulted in hospitalization. The estimated annual cost of asthma in the United States is approximately $16.1 billion, including an estimated $11.5 billion in direct costs such as asthma medications, physician office visits, emergency room visits and hospitalizations.

Chronic obstructive pulmonary disease (COPD) is a term referring to two lung diseases, chronic bronchitis and emphysema, that are characterized by obstruction to airflow that interferes with normal breathing. Smoking is the primary risk factor for COPD. Approximately 80 to 90 percent of COPD deaths are caused by smoking. Other risk factors of COPD include air pollution, second-hand smoke, history of childhood respiratory infections and heredity. According to the American Lung Association, in 2004, the cost to the United States for COPD was approximately $37.2 billion, including healthcare expenditures of $20.9 billion in direct health care expenditures, $7.4 billion in indirect morbidity costs and $8.9 billion in indirect mortality costs.

Lung cancer is the leading cancer killer in both men and women in the United States causing more deaths than the next three most common cancers combined (colon, breast and prostate). Approximately 170,000 deaths from lung cancer will occur in the United States during 2007. An estimated 360,000 Americans are living with lung cancer. During 2007 an estimated 180,000 new cases of lung cancer will be diagnosed. The expected 5-year survival rate for all patients in whom lung cancer is diagnosed is 15.5 percent compared to 64.8 percent for colon, 89 percent for breast and 99.9 percent for prostate cancer. Roughly 50% of this newly diagnosed population present with an obstructive lesion in the trachea or bronchus, thus affecting their ability to breath. In addition to these lesions, a significant number of patients suffer from benign airway lesions including, but are not limited to, granulomatous responses. These tissues typically form in response to a superficial airway injury and can quickly become life-threatening airway blockages. Both lesion types are difficult to manage and are very often fatal.

Traditionally, approaches to managing obstructive airway lesions have included surgery and endoscopic cauterizing therapy. Surgical outcomes are poor with very high complication rates.

Endoscopic alternatives are associated with risks. Cautery devices include, for example, argon plasma coagulation, radio frequency ablation, or laser ablation devices. Due to the nature of the lesions, interventional endoscopic procedures are usually performed under general sedation in an operating room setting, where the patient is connected to a ventilator. However during the procedure, the patient is typically removed from the ventilator. If a patient remains on the ventilator during use of the cautery device, the cautery device can ignite the oxygen-rich environment resulting in a fire in the patient's airway. The doctors must disconnect the ventilator while quickly performing the ablation procedure. The removal and return of the patient to the ventilator continues until the patient's oxygen saturation reaches a low warning level. As a result, the patient can suffer transient hypoxia several times throughout the procedure. Thus, the patient is subjected to additional risks during cautery therapy.

The sedation of the patient presents another obstacle when attempting to manage the blocking airway lesions using traditional methods. In particular, the patient must be sedated to the point where the gag and coughing reflexes are suppressed. However, these reflexes should not be suppressed to the degree that induces pulmonary paralysis.

Thus the medical industry would benefit from a new therapy for use with lung tissues, where the patient is not susceptible to an airway fire such that the patient can remain on the ventilator during the entire procedure and will not suffer the procedural and anesthesia risks associated with transient hypoxia.

A number of other conditions affecting the airway and respiratory tissues are also prevalent. Many of these are associated with conventional treatments that involve potential complications and expense. New, effective treatments for these conditions are also needed.

### SUMMARY OF THE INVENTION

The present invention overcomes the drawbacks of the prior art by using cryogenic spray to treat tissues in the thoracic cavity including, but are not limited to, lung tissue, pleura, and chest wall tissue. Tissues in the thoracic cavity include, but are not limited to, normal, abnormal, damaged, diseased, or unwanted lung tissue, pleura, and chest wall tissue as well as to induce a systemic immune and antimetastatic response.

In some embodiments, the present invention provides methods for treating lung tissue (including the external and internal portions of the lung), pleural tissue, and/or chest wall tissue. Such methods may comprise contacting the tissue with a cryogen, for example, with a liquefied gas such as liquid nitrogen. In some embodiments, the tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm is sufficiently depressed from normal body temperature to initiate a desired response. Suitable examples of temperatures may include, but are not limited to, from about 4° C. to about the boiling point of the cryogen. In this method and in any of the methods disclosed below, a target tissue may be contacted with cryogen, or an isotherm may be created in proximity to the target tissue, a plurality of times (e.g., two, three, four, five, six, seven, eight, nine, ten, etc. times). In any method where target tissue is contacted with cryogen, or an isotherm created in proximity to the target tissue, a plurality of times, the period of time between contacting or creation of the isotherm may be from about 1 second to about 10 minutes.

In some methods for treating lung tissue, pleural tissue, and/or chest wall tissue, a tissue to be treated is contacted with cryogen for a period of time sufficient to initiate a desired response. In some embodiments, a tissue to be treated is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue.

Methods for treating lung tissue, pleural tissue, and/or chest wall tissue will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating a lesion in lung tissue, pleural tissue, and/or chest wall tissue. Such methods may comprise contacting the tissue comprising the lesion with a cryogen, for example, with a liquefied gas such as liquid nitrogen. In some embodiments, the tissue comprising the lesion is not contacted directly with the cryogen. Instead, cryogen is used to create an isotherm in proximity to the tissue comprising the lesion to be treated. The temperature of the isotherm can be adjusted by controlling the rate at which cryogen is delivered through the distal end of the catheter.

The temperature of the isotherm may be sufficiently depressed from normal body temperature to generate the desired response. Suitable examples of temperatures may include, but are not limited to, from about 4° C. to about the boiling point of the cryogen.

In some methods of treating a lesion in lung tissue, pleural tissue, and/or chest wall tissue, the lesion and/or tissue comprising the lesion to be treated may be contacted with cryogen for a period of time sufficient to initiate a response in and/or freeze the lesion and/or tissue comprising the lesion. Alternatively, the lesion and/or tissue comprising the lesion to be treated may be in proximity to an isotherm having a temperature below the freezing point of the tissue for a period of time sufficient to initiate a response in and/or freeze the tissue. In some methods, the lesion and/or tissue comprising the lesion will not be frozen. The temperature of the lesion and/or tissue comprising the lesion may be reduced. In some embodiments, the temperature of the lesion and/or tissue comprising the lesion may be reduced sufficiently to stimulate cellular necrosis, for example, in the lesion.

Methods of treating a lesion in lung tissue, pleural tissue, and/or chest wall tissue will typically involve delivering cryogen from a cryogen source to a site to be treated. For example, a proximal end of a catheter may be connected to a cryogen source and a distal end of the catheter may be guided to a lesion and/or a tissue comprising a lesion to be treated using a guiding device and cryogen flows from the source through the distal end to the tissue. A guiding device may comprise a video camera and the distal end of the guiding device and the catheter may be guided to the lesion and/or tissue comprising the lesion by observing the distal end of the catheter and/or guiding device on a video monitor.

In some embodiments, a lesion may comprise unwanted tissue. In methods of this kind, the lesion and/or tissue comprising the lesion may be frozen, for example, by contacting the lesion and/or tissue comprising the lesion with cryogen or by keeping the tissue in proximity with an isotherm of sufficiently low temperature. In some methods, the lesion and/or tissue comprising the lesion will not be frozen. The temperature of the lesion and/or tissue comprising the lesion may be reduced sufficiently to stimulate cellular necrosis, for example, in the lesion.

In some embodiments, the present invention provides methods for freezing lung tissue, pleura, and/or chest wall tissue. Such methods may comprise contacting the lung tissue, pleura, and/or chest wall tissue with a cryogen, for example, with a liquefied gas such as liquid nitrogen. In some embodiments, the lung tissue, pleura, and/or chest wall tissue is not contacted directly with the cryogen. Instead, cryogen is used to create an isotherm in proximity to the lung tissue, pleura, and/or chest wall tissue. In some methods of treating lung tissue, pleura, and/or chest wall tissue the lung tissue, pleura, and/or chest wall tissue may be contacted with cryogen for a period of time sufficient to initiate a response in it. Alternatively, the lung tissue, pleura, and/or chest wall tissue may be in proximity to an isotherm having a temperature below the freezing point of the tissue for a period of time sufficient to initiate a response in and/or freeze the tissue. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 0° C. to about the boiling point of the cryogen.

Methods of freezing lung tissue, pleura, and/or chest wall tissue will typically involve delivering cryogen from a cryogen source to a site to be frozen. For example, a proximal end of a catheter may be connected to a cryogen source and a distal end of the catheter may be guided to lung tissue, pleura, and/or chest wall tissue to be frozen using a guiding device and cryogen flows from the source through the distal end to the site to be frozen. A guiding device may comprise a video camera and the distal end of the guiding device and the catheter may be guided to the tissue to be frozen by observing the distal end of the catheter and/or guiding device on a video monitor.

In some embodiments, the present invention provides methods of treating an infection in a lung. Examples of infections that can be treated include, but are not limited to, bacterial infections (e.g., pneumonia), viral infections, and mycobacterial infections (e.g., tuberculosis). Such methods may comprise contacting the infected lung tissue with a cryogen, for example, with a liquefied gas such as liquid nitrogen. In some embodiments, the infected lung tissue is not contacted directly with the cryogen. Instead, cryogen is used to create an isotherm in proximity to the infected lung tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating an infection in a lung, infected lung tissue to be treated is contacted with cryogen for a period of time sufficient to initiate a response in and/or freeze the infected lung tissue. Alternatively, the infected lung tissue may be in proximity to an isotherm having a temperature below the freezing point of the tissue for a period of time sufficient to initiate a response in and/or freeze the tissue. In some methods, the infected lung tissue will not be frozen. The temperature of the infected lung tissue may be reduced. In some embodiments, the infected lung tissue may be undergoing an inflammatory immune response and the temperature of the infected lung tissue may be reduced sufficiently to dampen the inflammatory response. The temperature of the isotherm may be sufficiently depressed from normal body temperature to generate the desired response. Suitable examples of temperatures may include, but are not limited to, from about 4° C. to about the boiling point of the cryogen.

Methods of treating infected lung tissue will typically involve delivering cryogen from a cryogen source to a site to be treated. For example, a proximal end of a catheter may be connected to a cryogen source and a distal end of the catheter may be guided to infected lung tissue to be treated using a guiding device and cryogen flows from the source through the distal end to the tissue. A guiding device may comprise a video camera and the distal end of the guiding device and the catheter may be guided to the infected lung tissue by observing the distal end of the catheter and/or guiding device on a video monitor.

In some embodiments, the present invention provides methods of treating unwanted tissue in a lung. Such methods may comprise contacting the unwanted tissue with a cryogen, for example, with a liquefied gas such as liquid nitrogen. In some embodiments, the unwanted tissue is not contacted directly with the cryogen. Instead, cryogen is used to create an isotherm in proximity to the unwanted tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating unwanted tissue in a lung, unwanted tissue to be treated is contacted with cryogen for a period of time sufficient to initiate a response in and/or freeze the tissue. Alternatively, the unwanted tissue may be in proximity to an isotherm having a temperature below the freezing point of the tissue for a period of time sufficient to initiate a response in and/or freeze the tissue. In some embodiments, the temperature of the unwanted tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue. The temperature of the unwanted tissue may be reduced sufficiently to stimulate cellular necrosis.

Methods of treating unwanted tissue in a lung will typically involve delivering cryogen from a cryogen source to a site to be treated. For example, a proximal end of a catheter may be connected to a cryogen source and a distal end of the catheter may be guided to unwanted tissue to be treated using a guiding device and cryogen flows from the source through the distal end to the unwanted tissue. A guiding device may comprise a video camera and the distal end of the guiding device and the catheter may be guided to the unwanted tissue by observing the distal end of the catheter and/or guiding device on a video monitor.

In some embodiments, the present invention provides methods of modulating an immune response in lung tissue, methods of inducing a systemic immune, and/or methods of inducing an antimetastatic response. Such methods may comprise contacting the tissue with a cryogen, for example, with a liquefied gas such as liquid nitrogen. In some embodiments, the tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In methods of modulating an immune response in lung tissue, methods of inducing a systemic immune, and/or methods of inducing an antimetastatic response, a tissue to be treated is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue. In some methods, the immune response in the tissue is reduced and the tissue is not frozen. In some methods, the immune response in the tissue is increased and the tissue is frozen.

Methods of modulating an immune response in lung tissue, methods of inducing a systemic immune, and/or methods of inducing an antimetastatic response will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of stimulating cartilage growth. Such methods typically entail injuring the cartilage with a cryogen, for example, with a liquefied gas such as liquid nitrogen under conditions resulting in stimulation of chondrogenesis. In some embodiments, the cartilage is not contacted directly with the cryogen. Instead, cryogen is used to create an isotherm in proximity to the cartilage to be injured. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of stimulating cartilage growth, cartilage is contacted with cryogen for a period of time sufficient to initiate a response in and/or freeze the cartilage. Alternatively, the cartilage may be in proximity to an isotherm having a temperature below the freezing point of the cartilage for a period of time sufficient to initiate a response in and/or freeze the cartilage. In some embodiments, the temperature of the cartilage is reduced but the cartilage is not frozen. This can be accomplished by creating an isotherm in proximity to the cartilage to be treated, wherein the temperature of the isotherm is below that of the cartilage and maintaining the cartilage in proximity to the isotherm for a period of time sufficient to reduce the temperature of the cartilage. In some embodiments, cartilage is contacted with cryogen for a period of time sufficient to damage a portion of the cartilage. In some embodiments, a plurality of isotherms may be created in proximity to the cartilage to stimulate chondrogenesis. For example, a first isotherm may be created at a first temperature and the cartilage maintained in proximity to the first isotherm. The first isotherm may be removed and a second isotherm which may be at the same or different temperature may created and the cartilage maintained in proximity to the second isotherm. A period of time may elapse between removal of the first isotherm and creation of the second isotherm. Any number of isotherms may be created and their temperatures may be the same or different. A period of time may elapse between the removal of one isotherm and the creation of a second or a second may be created by modifying (for example, by increasing or decreasing the temperature) a first with no period of time between. The temperature of the isotherm may be sufficiently depressed from normal body temperature to generate the desired response. Suitable examples of temperatures may include, but are not limited to, from about 4° C. to about the boiling point of the cryogen.

Methods of stimulating cartilage growth will typically involve delivering cryogen from a cryogen source to a site to be treated. For example, a proximal end of a catheter may be connected to a cryogen source and a distal end of the catheter may be guided to cartilage to be treated using a guiding device and cryogen flows from the source through the distal end to the cartilage. A guiding device may comprise a video camera and the distal end of the guiding device and the catheter may be guided to the tissue by observing the distal end of the catheter and/or guiding device on a video monitor.

In some embodiments, the present invention provides methods of treating damaged cartilage in a subject in need thereof. Damaged cartilage includes, but is not limited to, torn cartilage, chronically inflamed cartilage, and/or diminished cartilage. Thus, methods of the invention may be used to treat cartilage that is physically damaged or chronically inflamed prior to application of the cryogen. Such methods may include identifying a tissue of the subject comprising damaged cartilage and injuring the damaged cartilage with a cryogen, for example, with a liquefied gas such as liquid nitrogen under conditions resulting in stimulation of chondrogenesis. Tissue comprising damaged cartilage may be identified using any technique known in the art, for example, visually inspecting the tissue by arthroscopy or imaging the tissue (e.g., with magnetic resonance imaging (MRI), ultrasound, or computerized axial tomography scan (CAT scan or CT scan)). In some embodiments, the cartilage is not contacted directly with the cryogen. Instead, cryogen is used to create an isotherm in proximity to the cartilage to be injured. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating damaged cartilage in a subject in need thereof, cartilage is contacted with cryogen for a period of time sufficient to initiate a response in and/or freeze the cartilage. Alternatively, the cartilage may be in proximity to an isotherm having a temperature below the freezing point of the cartilage for a period of time sufficient to initiate a response in and/or freeze the cartilage. In some embodiments, the temperature of the cartilage is reduced but the cartilage is not frozen. This can be accomplished by creating an isotherm in proximity to the cartilage to be treated, wherein the temperature of the isotherm is below that of the cartilage and maintaining the cartilage in proximity to the isotherm for a period of time sufficient to reduce the temperature of the cartilage. In some embodiments, cartilage is injured with cryogen for a period of time sufficient to damage a portion of the cartilage. In some embodiments, a plurality of isotherms may be created in proximity to the cartilage to stimulate chondrogenesis. For example, a first isotherm may be created at a first temperature and the cartilage maintained in proximity to the first isotherm. The first isotherm may be removed and a second isotherm which may be at the same or different temperature may created and the cartilage maintained in proximity to the second isotherm. A period of time may elapse between removal of the first isotherm and creation of the second isotherm. The temperature of the isotherm may be sufficiently depressed from normal body temperature to generate the desired response. Suitable examples of temperatures may include, but are not limited to, from about 4° C. to about the boiling point of the cryogen.

Methods of treating damaged cartilage in a subject in need thereof will typically involve delivering cryogen from a cryogen source to a site to be treated. For example, a proximal end of a catheter may be connected to a cryogen source and a distal end of the catheter may be guided to cartilage to be treated using a guiding device and cryogen flows from the source through the distal end to the cartilage. A guiding device may comprise a video camera and the distal end of the guiding device and the catheter may be guided to the tissue by observing the distal end of the catheter and/or guiding device on a video monitor.

In some embodiments, the present invention provides methods of transplanting tissue to a subject in need thereof. Such methods may comprise contacting tissue at a selected position in the subject with a cryogen; and attaching a tissue to be transplanted to the cryogen-treated selected position. Such methods may comprise contacting the tissue with a cryogen, for example, with a liquefied gas such as liquid nitrogen. In some embodiments, the tissue at a selected position in the subject is not contacted directly with the cryogen. Instead, cryogen is used to create an isotherm in proximity to the tissue to be treated. The temperature of the isotherm can be adjusted by controlling the rate at which cryogen is delivered through the distal end of the catheter The temperature of the isotherm may be sufficiently depressed from normal body temperature to generate the desired response. Suitable examples of temperatures may include, but are not limited to, from about 4° C. to about the boiling point of the cryogen.

In some methods of transplanting tissue to a subject in need thereof, a tissue at a selected position in the subject is contacted with cryogen for a period of time sufficient to initiate a response in and/or freeze the tissue. Alternatively, the tissue at a selected position in the subject may be in proximity to an isotherm having a temperature below the freezing point of the tissue for a period of time sufficient to initiate a response in and/or freeze the tissue. In some embodiments, the temperature of the tissue at a selected position in the subject is reduced but the tissue is not frozen. This can be accomplished by creating an isotherm in proximity to the tissue at a selected position in the subject to be treated, wherein the temperature of the isotherm is below that of the tissue and maintaining the tissue in proximity to the isotherm for a period of time sufficient to reduce the temperature of the tissue.

Methods of transplanting tissue to a subject in need thereof will typically involve delivering cryogen from a cryogen source to a site to be treated. For example, a proximal end of a catheter may be connected to a cryogen source and a distal end of the catheter may be guided to a tissue at a selected position in the subject to be treated using a guiding device and cryogen flows from the source through the distal end to the tissue. A guiding device may comprise a video camera and the distal end of the guiding device and the catheter may be guided to the tissue at a selected position in the subject by observing the distal end of the catheter and/or guiding device on a video monitor.

In some embodiments, the present invention provides methods of treating chronic bronchitis in a subject in need of such treatment. Such methods may comprise contacting mucus producing cells in the lung with a cryogen, for example, with a liquefied gas such as liquid nitrogen, for a period of time sufficient to initiate a response in and/or freeze the mucus producing cells. Such methods may also include identifying mucus producing cells. Mucus producing cells may be identified using any technique known in the art, for example, by tissue biopsy, ultrasound, confocal microscopy or other imaging techniques. In some embodiments, the mucus producing cells are not contacted directly with the cryogen. Instead, cryogen is used to create an isotherm in proximity to the mucus producing cells. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating chronic bronchitis, mucus producing cells are contacted with cryogen for a period of time sufficient to initiate a response in and/or freeze the cells. Alternatively, the cells may be in proximity to an isotherm having a temperature below the freezing point of the cells for a period of time sufficient to initiate a response in and/or freeze the cells. In some embodiments, the temperature of the cells is reduced but the cells are not frozen. This can be accomplished by creating an isotherm in proximity to the cells, wherein the temperature of the isotherm is below that of the cells and maintaining the cells in proximity to the isotherm for a period of time sufficient to reduce the temperature of the cells. The temperature of the cells may be reduced sufficiently to stimulate cellular necrosis of the mucus producing cells.

Methods of treating chronic bronchitis in a subject will typically involve delivering cryogen from a cryogen source to a site to be treated, i.e., mucus producing cells. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating emphysema in a subject in need thereof. Such methods may comprise contacting lung tissue in the subject with a cryogen, for example, with a liquefied gas such as liquid nitrogen. In some embodiments, the tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter The temperature of the isotherm may be sufficiently depressed from normal body temperature to generate the desired response. Suitable examples of temperatures may include, but are not limited to, from about 4° C. to about the boiling point of the cryogen.

In some methods of treating emphysema, a tissue to be treated is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue.

Methods of treating emphysema will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating bronchiectasis in a subject in need thereof. Such methods typically comprise contacting lung tissue in the subject with a cryogen, for example, with a liquefied gas such as liquid nitrogen. Such methods may also comprise identifying a portion of the lung of the subject comprising damaged bronchial tissues. Identification may be accomplished using any technique known to those skilled in the art, for example, by visual observation, by imaging technologies such as ultrasound, MRI, and CT or by any other method known in the art and may be performed before, during, and/or after application of cryogen. In some embodiments, the tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating bronchiectasis, a tissue to be treated (for example, a tissue comprising cartilage) is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue.

Methods of treating bronchiectasis will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating asthma in a subject in need thereof. Such methods typically comprise contacting lung tissue in the subject with cryogen for a period of time sufficient to initiate a response in and/or freeze smooth muscle tissue. Any suitable cryogen may be used, for example, a liquefied gas such as liquid nitrogen. In some embodiments, the tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating asthma, a tissue to be treated (for example, a tissue comprising smooth muscle) is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the tissue is reduced but the tissue is not frozen. This can be accomplished by creating an isotherm in proximity to the tissue to be treated, wherein the temperature of the isotherm is below that of the tissue and maintaining the tissue in proximity to the isotherm for a period of time sufficient to reduce the temperature of the tissue.

Methods of treating asthma will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating or relieving a stricture in an airway of a subject in need thereof. Such methods typically comprise contacting the stricture with a cryogen for a period of time sufficient to initiate a response in and/or freeze the stricture. Any suitable cryogen may be used, for example, a liquefied gas such as liquid nitrogen. In some embodiments, the stricture is not contacted directly with the cryogen. Instead, cryogen is used to create an isotherm in proximity to the stricture to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating or relieving a stricture in an airway, a stricture to be treated is contacted with cryogen for a period of time sufficient to initiate a response in and/or freeze the stricture. Alternatively, the tissue may be in proximity to an isotherm having a temperature below the freezing point of the tissue for a period of time sufficient to initiate a response in and/or freeze the stricture. In some embodiments, the temperature of the stricture is reduced but the stricture is not frozen. This can be accomplished by creating an isotherm in proximity to the stricture to be treated, wherein the temperature of the isotherm is below that of the stricture and maintaining the stricture in proximity to the isotherm for a period of time sufficient to reduce the temperature of the stricture.

Methods of treating or relieving a stricture in an airway will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating a benign or malignant tumor or lesion and neoplastic disease in a lung of a subject in need thereof. Such methods typically comprise contacting lung tissue in the subject comprising a benign or malignant tumor or lesion and/or neoplastic disease with a cryogen for a period of time sufficient to initiate a response in and/or freeze the benign or malignant tumor or lesion and/or neoplastic tissue. Any suitable cryogen may be used, for example, a liquefied gas such as liquid nitrogen. Any type of benign or malignant tumor or lesion and/or neoplastic disease may be treated. In some embodiments, the benign or malignant tumor or lesion and/or neoplastic tissue is selected from a group consisting of small cell carcinomas, non-small cell carcinomas, hamartoma, and mesothelioma. In some embodiments, the lung tissue comprising a benign or malignant tumor or lesion and/or neoplastic tissue is not contacted directly with the cryogen. Instead, cryogen is used to create an isotherm in proximity to the lung tissue comprising a benign or malignant tumor or lesion and/or neoplastic tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating a benign or malignant tumor or lesion and/or neoplastic disease in a lung, lung tissue comprising a benign or malignant tumor or lesion and/or neoplastic tissue is contacted with cryogen for a period of time sufficient to initiate a response in and/or freeze the benign or malignant tumor or lesion and/or neoplastic tissue. Non-affected tissue adjacent to the benign or malignant tumor or lesion and/or neoplastic disease tissue may or may not be frozen. Alternatively, the lung tissue comprising a benign or malignant tumor or lesion and/or neoplastic disease tissue may be in proximity to an isotherm having a temperature below the freezing point of the tissue for a period of time sufficient to initiate a response in and/or freeze the tissue. In some embodiments, the temperature of the benign or malignant tumor or lesion and/or neoplastic tissue is reduced but the benign or malignant tumor or lesion and/or neoplastic tissue is not frozen. This can be accomplished by creating an isotherm in proximity to the tissue to be treated, wherein the temperature of the isotherm is below that of the benign or malignant tumor or lesion and/or neoplastic tissue and maintaining the benign or malignant tumor or lesion and/or neoplastic tissue in proximity to the isotherm for a period of time sufficient to reduce the temperature of the benign or malignant tumor or lesion and/or neoplastic tissue.

Methods of treating a benign or malignant tumor or lesion and/or neoplastic disease in a lung will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating pleurisy in a subject in need thereof. Such methods typically comprise contacting a portion of a pleura of the subject with a cryogen. In some embodiments, contacting may comprise using laparoscopy to access the pleura. In some embodiments, methods may include applying suction to remove gaseous cryogen. In some embodiments, methods may comprise identifying inflamed pleural tissue. Identification may be accomplished using any technique known to those skilled in the art, for example, by visual observation, by imaging technologies such as ultrasound, MRI, and CT or by any other method known in the art and may be performed before, during, and/or after application of cryogen. Any suitable cryogen may be used, for example, a liquefied gas such as liquid nitrogen. In some embodiments, the pleura is not contacted directly with the cryogen. Instead, cryogen is used to create an isotherm in proximity to the portion of the pleura to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating pleurisy, a pleura may be contacted with cryogen for a period of time sufficient to initiate a response in and/or freeze a portion of the pleura. Tissue adjacent to the portion of the pleura to be treated may or may not be frozen. Alternatively, the portion of the pleura to be treated may be in proximity to an isotherm having a temperature below the freezing point of the portion for a period of time sufficient to initiate a response in and/or freeze the portion. In some embodiments, the temperature of the portion of the pleura to be treated is reduced but the portion is not frozen. This can be accomplished by creating an isotherm in proximity to the portion to be treated, wherein the temperature of the isotherm is below that of the portion and maintaining the portion in proximity to the isotherm for a period of time sufficient to reduce the temperature of the portion of the pleura to be treated.

Methods of treating pleurisy will typically involve delivering cryogen from a cryogen source to a site to be treated. For example, a proximal end of a catheter may be connected to a cryogen source and a distal end of the catheter may be guided to a portion of a pleura to be treated using a guiding device and cryogen flows from the source through the distal end to the portion to be treated. A guiding device may comprise a video camera and the distal end of the guiding device and the catheter may be guided to the portion of the pleura to be treated by observing the distal end of the catheter and/or guiding device on a video monitor.

In some embodiments, the present invention provides methods of treating occupational lung disease in a subject in need thereof. Such methods typically comprise identifying tissue in the lung of the subject affected by occupational lung disease and contacting the affected tissue with cryogen for a period of time sufficient to initiate a response in and/or freeze the tissue. Any type of affected tissue may be treated, for example, the tissue may comprise one or more conditions selected from a group consisting of reticular nodules, reticular micronodules, macronodules and fibrous tissue. Identification may be accomplished using any technique known to those skilled in the art, for example, by visual observation, by imaging technologies such as ultrasound, MRI, and CT or by any other method known in the art and may be performed before, during, and/or after application of cryogen. Any suitable cryogen may be used, for example, a liquefied gas such as liquid nitrogen. In some embodiments, the affected tissue is not contacted directly with the cryogen. Instead, cryogen is used to create an isotherm in proximity to the affected tissue. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating occupational lung disease, affected tissue may be contacted with cryogen for a period of time sufficient to initiate a response in and/or freeze all or a portion of the affected tissue. Tissue adjacent to the portion to be treated may or may not be frozen. Alternatively, the portion of the affected tissue to be treated may be in proximity to an isotherm having a temperature below the freezing point of the affected tissue for a period of time sufficient to initiate a response in and/or freeze the portion of the affected tissue. In some embodiments, the temperature of the affected tissue to be treated is reduced but the affected tissue is not frozen. This can be accomplished by creating an isotherm in proximity to the affected tissue to be treated, wherein the temperature of the isotherm is below that of the affected tissue and maintaining the affected tissue in proximity to the isotherm for a period of time sufficient to reduce the temperature of the affected tissue to be treated.

Methods of treating occupational lung disease will typically involve delivering cryogen from a cryogen source to a site to be treated. For example, a proximal end of a catheter may be connected to a cryogen source and a distal end of the catheter may be guided to affected tissue to be treated using a guiding device and cryogen flows from the source through the distal end to the affected tissue to be treated. A guiding device may comprise a video camera and the distal end of the guiding device and the catheter may be guided to the affected tissue to be treated by observing the distal end of the catheter and/or guiding device on a video monitor.

In some embodiments, the present invention provides methods of treating pulmonary vascular diseases in a subject in need thereof. Such methods typically comprise contacting pulmonary vascular tissue with a cryogen for example, with a liquefied gas such as liquid nitrogen. Such methods may also comprise identifying diseased pulmonary vascular tissues. Identification may be accomplished using any technique known to those skilled in the art, for example, by visual observation, by imaging technologies such as ultrasound, MRI, and CT or by any other method known in the art and may be performed before, during, and/or after application of cryogen. In some embodiments, the diseased pulmonary vascular tissue is not contacted directly with the cryogen. Instead, cryogen is used to create an isotherm in proximity to the diseased pulmonary vascular tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating pulmonary vascular diseases, diseased pulmonary vascular tissue to be treated is contacted with cryogen for a period of time sufficient to initiate a response in and/or freeze the tissue. Alternatively, the diseased pulmonary vascular tissue may be in proximity to an isotherm having a temperature below the freezing point of the tissue for a period of time sufficient to initiate a response in and/or freeze the tissue. In some embodiments, the temperature of the diseased pulmonary vascular tissue is reduced but the tissue is not frozen. This can be accomplished by creating an isotherm in proximity to the diseased pulmonary vascular tissue to be treated, wherein the temperature of the isotherm is below that of the tissue and maintaining the tissue in proximity to the isotherm for a period of time sufficient to reduce the temperature of the tissue.

Methods of treating pulmonary vascular diseases will typically involve delivering cryogen from a cryogen source to a site to be treated. For example, a proximal end of a catheter may be connected to a cryogen source and a distal end of the catheter may be guided to a diseased pulmonary vascular tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating drug induced lung disease in a subject in need thereof. Such methods typically comprise contacting diseased lung tissue with a cryogen for example, with a liquefied gas such as liquid nitrogen. Such methods may also comprise identifying diseased lung tissue. Identification may be accomplished using any technique known to those skilled in the art, for example, by visual observation, by imaging technologies such as ultrasound, MRI, and CT or by any other method known in the art and may be performed before, during, and/or after application of cryogen. In some embodiments, the diseased lung tissue is not contacted directly with the cryogen. Instead, cryogen is used to create an isotherm in proximity to the diseased lung tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating drug induced lung disease, diseased lung tissue to be treated is contacted with cryogen for a period of time sufficient to initiate a response in and/or freeze the tissue. Alternatively, the diseased lung tissue may be in proximity to an isotherm having a temperature below the freezing point of the tissue for a period of time sufficient to initiate a response in and/or freeze the tissue. In some embodiments, the temperature of the diseased lung tissue is reduced but the tissue is not frozen. This can be accomplished by creating an isotherm in proximity to the diseased lung tissue to be treated, wherein the temperature of the isotherm is below that of the tissue and maintaining the tissue in proximity to the isotherm for a period of time sufficient to reduce the temperature of the tissue.

Methods of treating drug induced lung disease will typically involve delivering cryogen from a cryogen source to a site to be treated. For example, a proximal end of a catheter may be connected to a cryogen source and a distal end of the catheter may be guided to a diseased lung tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating acute respiratory distress syndrome in a subject in need thereof. Such methods typically comprise contacting lung tissue with a cryogen for example, with a liquefied gas such as liquid nitrogen. Such methods may also comprise identifying a subject having acute respiratory distress syndrome. Identification may be accomplished using any technique known to those skilled in the art, for example, by visual observation, by chest sounds, by imaging technologies such as ultrasound, MRI, and CT or by any other method known in the art and may be performed before, during, and/or after application of cryogen. In some embodiments, the lung tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating acute respiratory distress syndrome, lung tissue to be treated is contacted with cryogen for a period of time sufficient to initiate a response in and/or freeze the tissue. Alternatively, the lung tissue may be in proximity to an isotherm having a temperature below the freezing point of the tissue for a period of time sufficient to initiate a response in and/or freeze the tissue. In some embodiments, the temperature of the lung tissue is reduced but the tissue is not frozen. This can be accomplished by creating an isotherm in proximity to the lung tissue to be treated, wherein the temperature of the isotherm is below that of the tissue and maintaining the tissue in proximity to the isotherm for a period of time sufficient to reduce the temperature of the tissue.

Methods of treating acute respiratory distress syndrome will typically involve delivering cryogen from a cryogen source to a site to be treated. For example, a proximal end of a catheter may be connected to a cryogen source and a distal end of the catheter may be guided to a lung tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating interstitial and/or granulomatous diseases in a subject in need thereof. Such methods typically comprise contacting interstitial and/or granulomatous lung tissue with a cryogen for example, with a liquefied gas such as liquid nitrogen. Such methods may also comprise identifying a subject having interstitial and/or granulomatous diseases. Identification may be accomplished using any technique known to those skilled in the art, for example, by visual observation, by chest sounds, by imaging technologies such as ultrasound, MRI, and CT or by any other method known in the art and may be performed before, during, and/or after application of cryogen. In some embodiments, the lung tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating interstitial and/or granulomatous diseases, lung tissue to be treated is contacted with cryogen for a period of time sufficient to initiate a response in and/or freeze the tissue. Alternatively, the lung tissue may be in proximity to an isotherm having a temperature below the freezing point of the tissue for a period of time sufficient to initiate a response in and/or freeze the tissue. In some embodiments, the temperature of the lung tissue is reduced but the tissue is not frozen. This can be accomplished by creating an isotherm in proximity to the lung tissue to be treated, wherein the temperature of the isotherm is below that of the tissue and maintaining the tissue in proximity to the isotherm for a period of time sufficient to reduce the temperature of the tissue.

Methods of treating interstitial and/or granulomatous diseases will typically involve delivering cryogen from a cryogen source to a site to be treated. For example, a proximal end of a catheter may be connected to a cryogen source and a distal end of the catheter may be guided to a lung tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

In some embodiments, the present invention provides methods of treating exuberant granulation tissue in a subject in need thereof. Such methods typically comprise contacting exuberant granulation tissue with a cryogen for example, with a liquefied gas such as liquid nitrogen. Such methods may also comprise identifying a subject having exuberant granulation tissue. Identification may be accomplished using any technique known to those skilled in the art, for example, by visual observation, by chest sounds, by imaging technologies such as ultrasound, MRI, and CT or by any other method known in the art and may be performed before, during, and/or after application of cryogen. In some embodiments, the exuberant granulation tissue is not contacted directly with the cryogen. Instead, cryogen is used to create an isotherm in proximity to the tissue to be treated. The temperature of the isotherm can be adjusted by controlling the rate at which cryogen is delivered through the distal end of the catheter. The temperature of the isotherm may be sufficiently depressed from normal body temperature to generate the desired response. Suitable examples of temperatures may include, but are not limited to, from about 4° C. to about the boiling point of the cryogen.

In some methods of treating exuberant granulation tissue, tissue to be treated is contacted with cryogen for a period of time sufficient to initiate a response in and/or freeze the tissue. Alternatively, the tissue may be in proximity to an isotherm having a temperature below the freezing point of the tissue for a period of time sufficient to initiate a response in and/or freeze the tissue. In some embodiments, the temperature of the exuberant granulation tissue is reduced but the tissue is not frozen. This can be accomplished by creating an isotherm in proximity to the exuberant granulation tissue to be treated, wherein the temperature of the isotherm is below that of the tissue and maintaining the tissue in proximity to the isotherm for a period of time sufficient to reduce the temperature of the tissue.

Methods of treating exuberant granulation tissue will typically involve delivering cryogen from a cryogen source to a site to be treated. For example, a proximal end of a catheter may be connected to a cryogen source and a distal end of the catheter may be guided to a tissue to be treated using a guiding device and cryogen flows from the source through the distal end to the tissue. A guiding device may comprise a video camera and the distal end of the guiding device and the catheter may be guided to the tissue by observing the distal end of the catheter and/or guiding device on a video monitor.

The present invention also provides methods for treating distal airway diseases, comprising contacting the tissue in the distal airway passage with a cryogen or a non-cryogenic gas, or using the cryogen or the non-cryogenic gas to create an isotherm in proximity to the tissue.

In some methods, the present invention may further comprise administering an agent, for example, a therapeutic agent or a diagnostic agent using a single lumen catheter or a multiple lumen catheter (for example, a dual lumen catheter), wherein the therapeutic agent may be administered before, at the same time or after the delivery of the cryogen. The therapeutic agents include, but are not limited to, anticancer agents (for example cancer chemotherapeutic agents, biological response modifiers, vascularization inhibitors, hormone receptor blockers, or other agents that destroy or inhibit neoplasia or tumorigenesis), anti-fungal agents, anti-viral agents, including anti-retroviral agents, anti-microbial agents, anti-rheumatic agents, immunomodulatory agents, steroids or other anti-inflammatory agents, cytokine inhibitors, vasoconstrictors, mucolytics, including cells (for example, mucosal cells, fibroblasts, stem cells or genetically engineered cells) as well as genes and gene delivery vehicles like plasmids, viruses (e.g. adenoviral vectors), naked or complexed nucleic acids, for example, DNA, mRNA, etc.

In some embodiments, the therapeutic, diagnostic, or other agents can be delivered via injection or infusion using a needle catheter, for example a microneedle catheter; in addition to including direct application of drug into the lungs, by inhalation therapy using either pressurized metered dose inhalers (pMDI) or dry powder inhalers (DPI), intratracheal administration, and including, but are not limited to, inhalers, nebulizers (including jet or ultrasonic nebulizers) and other standard pulmonary delivery methods known in the art, for example, intratracheal inhalation or insufflation. To convey a sufficient dose of drug to the lungs, suitable drug carriers can be used. These include, but are not limited to, solid, liquid, or gaseous excipients, liposomes, nano- and microparticles, cyclodextrins, microemulsions, micelles, suspensions, or solutions. The use of microreservoir-type systems offers advantages such as high loading capacity and the possibility of controlling size and permeability, and thus of controlling the release kinetics of the drugs from the carrier systems. These systems make it possible to use relatively small numbers of vector molecules to deliver substantial amounts of a drug to the target.

In some embodiments of the invention, the method comprises delivering therapeutic agents without a cryogen (for example a non-cryogenic gas), including, but are not limited to, oxygen, room air and CO₂, wherein the lesion and/or tissue comprising the lesion to be treated is not frozen upon the contact of non-cryogenic gas.

In some embodiments, the method comprises treating a lesion and/or tissue comprising the lesion to be treated contacting with a non-cryogenic gas for a period of time sufficient to initiate a response in and/or without freezing the lesion and/or tissue comprising the lesion. Alternatively, the lesion and/or tissue comprising the lesion to be treated may be in proximity to an isotherm having a temperature above the freezing point of the tissue for a period of time sufficient to initiate a response in and/or without freezing the lesion and/or tissue comprising the lesion.

In another embodiment of the invention, the method comprises delivering therapeutic agents directly onto the lesion and/or tissue comprising the lesion to be treated with or without the cryogen or non-cryogenic gas.

### BRIEF DESCRIPTION OF THE DRAWINGS

For the purpose of illustrating the invention, there are shown in the drawings forms which are presently preferred; it being understood, that this invention is not limited to the precise arrangements and instrumentalities shown.

FIG. 1A depicts a bronchoscope inserted into the lung of a patient.

FIG. 1B is an enlarged view of FIG. 1A and shows the distal end of the bronchoscope and the catheter within bronchiole.

FIG. 2A depicts a close-up of the distal end of the bronchoscope, light source, camera, extra lumen, and lumen with catheter.

FIG. 2B depicts a close-up of the distal end of the bronchoscope, light source, camera, extra lumen, and lumen with catheter with a lateral opening for emitting directional cryogen spray.

FIG. 2C depicts a close-up of the distal end of the bronchoscope, light source, camera, extra lumen, and lumen with a catheter having a laterally-disposed, cone-shaped structure for directing cryogen spray.

FIG. 3 is a schematic view of an apparatus for use in respiratory cryosurgery.

FIG. 4 is a schematic view of an alternative apparatus for use in respiratory cryosurgery

FIG. 5 depicts the structure of a heated catheter.

FIG. 6 is a flowchart showing steps of a method according to an embodiment of the invention.

FIGS. 7A and 7B show a side-by-side comparison of the treatment site at 7 days after 4 cycles of 5 seconds (FIG. 7A) or 2 cycles of 5 seconds (FIG. 7B) of cryofrost. FIG. 7A shows re-epithelialization of healing ulcer with squamous metaplasia, organizing fibrosis of subepithelial layer with some necrosis and inflammation involving smooth muscle layer, and damage to glands including active inflammation and squamous metaplasia. The hyaline cartilage layer is damaged with reformation of cartilage on the outside (arrow). The extent of damage in terms of horizontal axis is about 7.5 mm; maximum depth of injury is about 2.6 mm. FIG. 7B shows an injury to the depth of the second cartilaginous plate, about 2.7 mm. The surface tissue shows squamous metaplasia and fibroblasts, not specifically localized to the cartilaginous damage. The length of horizontal injury is 9 mm.

FIG. 8A shows maximum depth of injury of four cycles of 5 seconds. Injury extends to adventitia, about 2.8 mm and about 15 mm long. There is an ulcer with acute inflammation obliterates smooth muscle and glands and some pus.

FIG. 8B shows deep and extensive damage resulting from 2 cycles of 5 seconds of cryofrost. A deep ulcer, neutrophils in cartilage, and some lung damage at about 3.9 mm (arrows) are evident. A histology induced tear occurs at about 3.9 mm.

FIG. 9 shows histologically the complete reepithelialization and normalization of the tissue at day 28, with the exception of the cartilaginous layers up to a depth of about 2.7 mm, which may need additional time to heal due to lack of vascularity.

FIG. 10 shows the treatment site after a 28 day healing period. There is some evidence of prior injury to hyaline cartilage and cartilage degeneration, but also evidence of chondrogenesis. The depth of injury of the cartilage plate is 2.4 mm.

FIG. 11 shows a histological section of cryofrost-treated tissue that is 3.9 mm long and 0.6 mm deep in the process of being sloughed off. The superimposed window shows a magnified view of the separation of cryo-ablated tissue from the underlying tissue.

FIG. 12 shows the catheter in close up orientation.

FIG. 13 shows the histological section of untreated mucosa 1 hour post CSA treatment.

FIG. 14 shows the histological section of treated mucosa 1 hour post CSA treatment.

FIG. 15 shows the histological section of untreated airway mucosa 12 days post CSA treatment.

FIG. 16 shows the histological section of treated mucosa 12 days post CSA treatment.

FIG. 17 shows a cross section of airways 106 days post CSA treatment.

FIG. 18 shows an overgrown stent prior to cryospray treatment.

FIG. 19 shows the same stent as FIG. 18 after treatment.

FIG. 20 shows a chest X-ray of the patient treated for stent overgrowth using cryospray.

### DETAILED DESCRIPTION

It will be appreciated that the following description is intended to refer to embodiments of a cryosurgical apparatus for use in the methods of the present invention and is not intended to define or limit the invention, other than in the appended claims.

The present invention provides a system adapted to deliver materials to a target tissue. Any type of material known to one of skill in the art can be used, for example, a cryogen, therapeutic agents, diagnostic agents or any combination of such, etc. The materials can be selected from any one of solid, liquid, gas or liquefied gas or any combination of such. The target tissue can include, but is not limited to, any portion of the lung including proximal to distal airways and parenchyma. The distal airway may be defined anatomically as the region of the respiratory system including the terminal bronchioles through alveoli.

The present invention also provides a system adapted for delivering cryogen or using the cryogen to create an isotherm in proximity to a target tissue.

The present invention also provides a cryosurgery system comprising a cryogen delivering apparatus configured to deliver a cryogen or using the cryogen to create an isotherm in proximity to the target tissue.

One embodiment of the invention further comprises a system wherein a visualization (direct or indirect) apparatus configured to provide visualization of the target tissue during the delivery a cryogen or while using the cryogen to create an isotherm in proximity to the target tissue.

In one embodiment of the invention, the visualization apparatus and the cryogen delivery apparatus are constructed and arranged to be operationally unintegrated and physically spaced with respect to each other during the delivery of a cryogen or while using the cryogen to create an isotherm in proximity to the target tissue.

In another embodiment of the invention, the system may comprise a controller communicatively coupled to the regulation apparatus configured to control release of cryogen into the cryogen delivery apparatus.

In another embodiment of the invention, the visualization apparatus may comprise an external imaging system, such as, but are not limited to, a x-ray system, a computed tomography system, an ultrasound system and a magnetic resonance system.

In another embodiment of the invention, the system may comprise an insertable device that is separately guided from the cryogen delivery apparatus.

In one embodiment of the invention, the system is adapted to deliver therapeutic or diagnostic agents before the delivery of a cryogen or using the cryogen to create an isotherm in proximity to the target tissue.

In one embodiment of the invention, the system is adapted to deliver therapeutic or diagnostic agents at the same time as the delivery of a cryogen or using the cryogen to create an isotherm in proximity to the target tissue.

In one embodiment of the invention, the system is adapted to deliver therapeutic or diagnostic agents after the delivery of a cryogen or using the cryogen to create an isotherm in proximity to the target tissue.

The present invention provides materials and methods for treating tissue, which may be unwanted tissue, in the thoracic cavity. In some embodiments, the invention relates to a method of treating or preventing abnormal or pathogenic conditions in respiratory tissues. As used herein, the term “respiratory tissue(s)” includes those tissues of the respiratory airway(s) such as the trachea, bronchi, bronchioles, lungs, and all pleural tissues associated therewith. Such tissues can include muscle, blood vessels, lymphatic tissue, epithelial, mucosal and sub mucosal tissue, cartilage and other connective tissue of the thoracic cavity. Target tissues may be abnormal, diseased, damaged, or unwanted tissue. As used herein, the terms “target area”, “target tissue” and “tissue to be treated” refer to that portion of healthy, diseased, damaged or unwanted tissue to which a cryogen is, is to be, or has been applied.

Materials and Methods of the invention can be used to treat, for example, pseudo stratified ciliated columnar epithelium, smooth muscle, submucosal glands, cartilage and adventitia found in the airway as well as other tissues of the respiratory tract, airways, chest wall or pleural space. The methods can also be used to treat in the thoracic cavity and may be particularly useful for conditions relating to the controlled injury and ablation of respiratory tissues including, but are not limited to, pulmonary lesions and conditions, such as, for example, asthma, COPD, and histologically proven or suspected carcinomas of the trachea or bronchi, inoperable tumors and lesions based on the position of the tumors, patients unsuitable for a lung resection due to poor respiratory function, recurrence of a tumor following other modality of treatment, for example, radiotherapy, chemotherapy, lung resection or other endobronchial treatment (Nd:YAG laser, brachytherapy), intraluminal tumors with little external compression, microinvasive carcinoma, hemoptysis caused by visible benign or malignant lesion, and granulation tissue following lung transplantation or stent placement, extraction of foreign bodies, blood clots, mucous plugs and excessive or uncontrolled bleeding in the airway. The methods also include non-ablative therapies, including, but are not limited to, hemostasis, immunomodulation, chondrogenesis, pleurisy, tissue transplantation, etc. Further indications for which the methods can be suitable are described below.

The methods can be carried out with a catheter alone or in combination with a guiding device, such as an endoscope. A camera or other viewing device can also be used if visualization of the target tissue is desired and the tissue is not otherwise easily viewable. When an endoscope is used, it is also possible to deliver cryogen to the target tissue directly through an endoscope channel without a catheter.

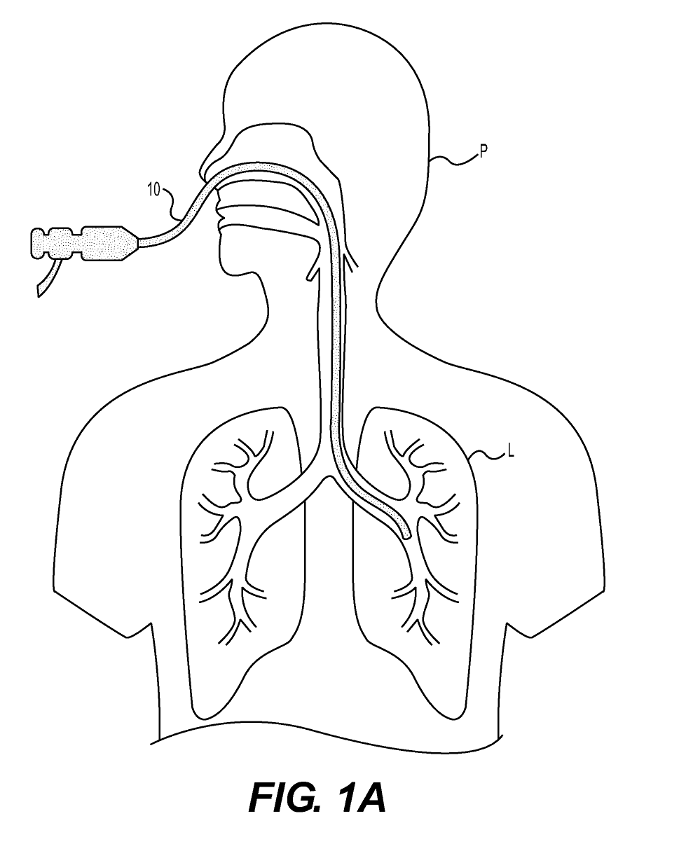

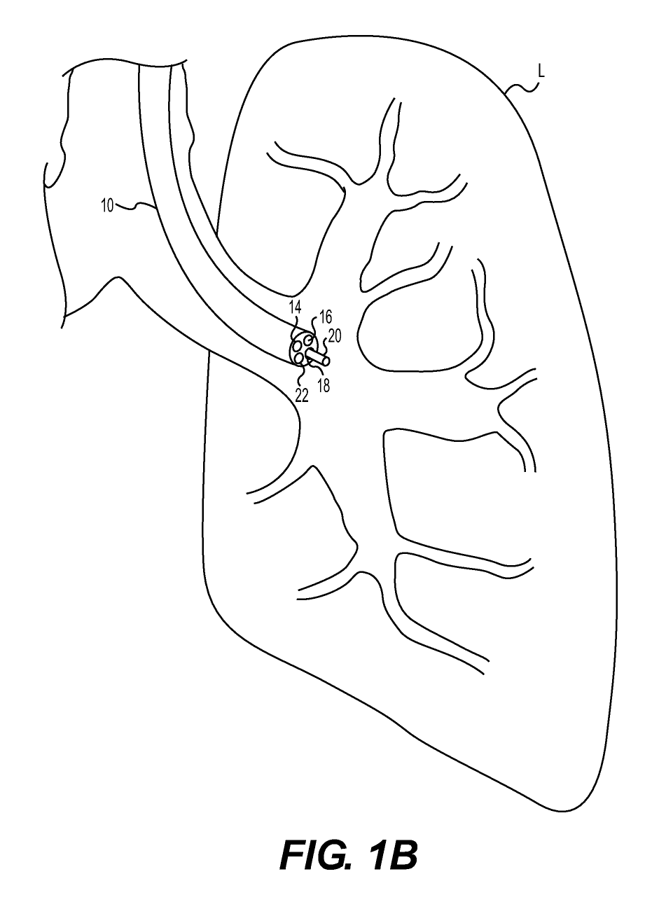

An apparatus for use in at least one method of the invention is shown in FIGS. 1A and 1B. The method can include performing cryospray ablation utilizing an endoscope, such as a bronchoscope, having a catheter inserted therethrough, where the bronchoscope and catheter can be inserted into a patient's upper respiratory tract or respiratory airways, including the trachea, bronchi or bronchioles. The catheter can be positioned to allow a cryogen fluid spray to be disposed adjacent a tissue to be sprayed (a.k.a. the target(ed) tissue). The tissue of the upper respiratory tract or respiratory airways of the patient can then be sprayed with a cryogen fluid spray. The rapid freezing and thawing of cryospray ablation evokes acute and chronic hemostatic effects and intracellular damage leading to regeneration of healthy tissue.

Alternatively, the pleural space or thoracic cavity may be accessed using laparoscopic techniques to allow cryotherapy of tissues that cannot be easily accessed through the airways. For example, a primary trocar may be used as a guiding device by inserting it in order to place a cannula adjacent target tissue, with or without a laparoscope to view internal structures. Other, secondary, trocars can provide for insertion of other instruments such as a cryospray catheter or biopsy forceps.

While not wishing to be bound by any particular theory, it is believed that Cryotherapy acts by initiating the process of tissue destruction at the frozen site. Cells exposed to flash freezing undergo necrosis secondary to direct cellular damage by ice crystals, as well as vascular and endothelial injury with consequent ischemia and subsequent infarction. If a cell is cooled slowly, the dehydration of the water from the extracellular space allows for an increase in the solute concentration that prevents intracellular freezing. If a cell is cooled rapidly, water does not have time to diffuse across the membrane, and ice crystals will form in the intracellular space. Slow thawing after freezing allows re-crystallization of the cytoplasm, which destroys intracellular organelles such as the mitochondria and may lead to mitochondria-regulated apoptosis. Thrombosis occurs almost immediately after a slow thaw, beginning a hemostatic cascade.

Although healthy tissue was sprayed in this study to determine feasibility and depth of injury, tumor cells have been shown to be more sensitive to cryotherapy than healthy cells, suggesting potential for palliation of benign and malignant blocking lesions. Furthermore, the cellular matrix, namely fat, connective tissue and cartilage have shown to be cryo-resistant, while the mucosa is ablated and subsequently regenerated.

Cryotherapy has been found to have a high rate of success in relieving airway obstruction caused by benign or malignant tumors, as well as granulation tissue and stenosis. Additionally, a synergistic response has been observed when cryotherapy is used as an adjuvant therapy to chemotherapy and radiotherapy.

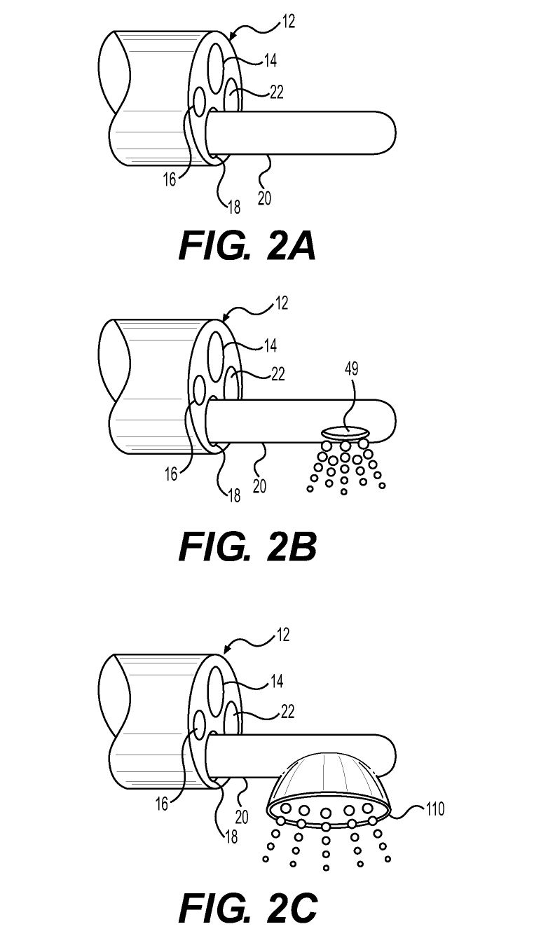

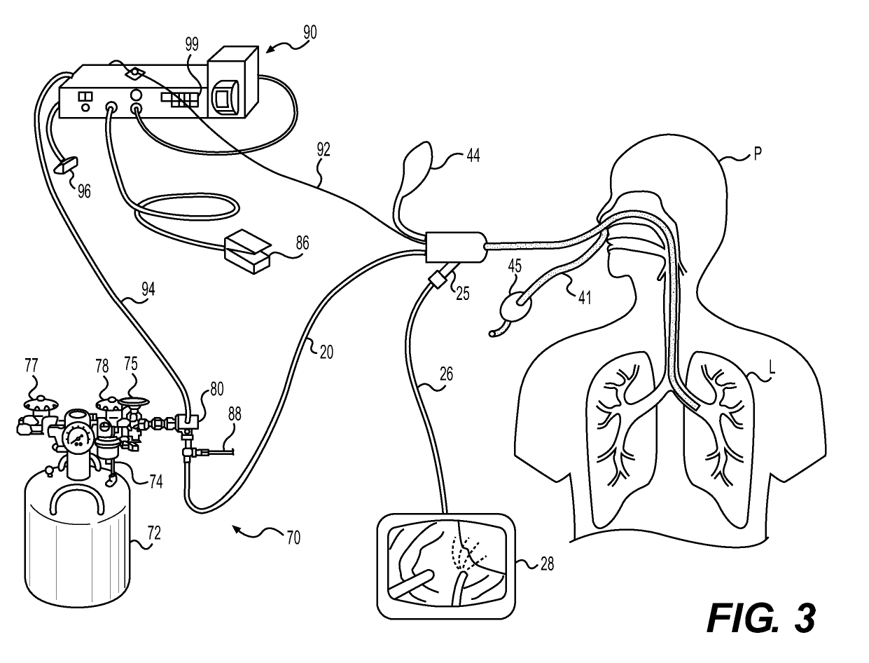

The method of the present invention can be performed using either a conventional therapeutic bronchoscope 10, as is illustrated in the drawings, or a smaller diagnostic bronchoscope to maximize patient comfort. Alternatively, a specially designed bronchoscope can be used. The distal end 12 of such a bronchoscope 10 is shown in FIGS. 2A, 2B, and 2C, showing an imaging camera lens 14, illuminating light 16, biopsy channel (bore or lumen) 18 with the catheter 20 therein, and an additional lumen 22. An additional catheter may be run through the additional lumen 22, for the delivery of therapeutic or diagnostic agents. The image picked up at the lens 14 is transferred via fiber optics to a monitoring camera 25 (FIG. 3) which sends TV signals via a cable 26 to a conventional monitor 28, where the procedure can be directly visualized by a physician. By virtue of this visualization, the surgeon is able to perform the cryosurgery with respect to respiratory tissues.

A catheter 20 can be disposed through the lumen 18. For some applications, the catheter can be a conventional polyimide catheter size 7 French of about 2-3 mm outside diameter. However, larger or smaller catheters and catheters made of other materials can be used. For example, an Olympus BF-1T160 therapeutic bronchoscope has a 2.8 mm working channel and a 60 cm working length. Any appropriate catheter sized to fit within the working channel (i.e., with a diameter of less than 2.8 mm) may be used if a BF-1T160 is employed. An Olympus BF-P160 has an insertion tube with an outer diameter of 4.9 mm and a working channel of length 60 cm and diameter of 2.0 mm. When using such a bronchoscope, a catheter with an outer diameter of less than 2.0 mm can be used, such as a 3, 4 or 5 French (having outer diameters of 1, 1.35 and 1.67 mm respectively). A therapeutic BF-XP60 fiberoptic bronchoscope and BF-XP40 fiberbroncoscope each have a working channel of 1.2 mm, which could accommodate a 3 French or smaller catheter. Smaller O.D. bronchoscopes and correspondingly smaller O.D. catheters may be utilized for treatment of respiratory tissues deep within the bronchioles, where larger devices may not fit without risk of puncture, abrasion or other unintended tissue damage. The catheters of the present invention can be of a thermoset or a thermoplastic material, may be manufactured from a combination of a number of materials including, but are not limited to, stainless steel, metal, nickel alloy, nickel-titanium alloy, hollow cylindrical stock, thermoplastics, high performance engineering resins, polymer, fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysulfone, nylon, perfluoro (propyl vinyl ether) (PFA), polyoxymethylene (POM), polybutylene terephthalate (PBT) or polyether block ester. The catheter is manufactured so as to maintain the desired level of flexibility and torqueability according to multiple embodiments of the current invention.

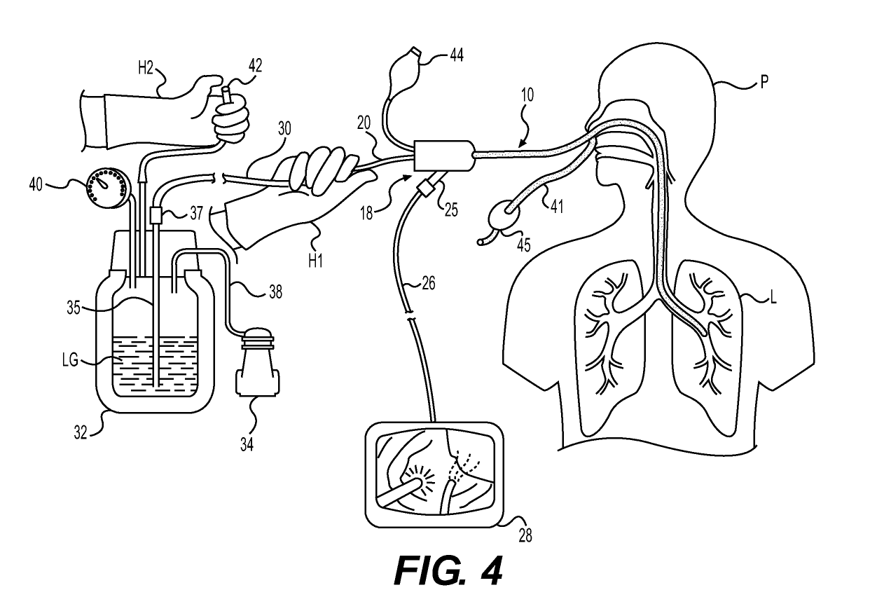

The catheter 20 may protrude from the distal end 12 (i.e., the end first inserted into the respiratory tract or respiratory airways) of the endoscope 10 and may extend to the proximal end 30 (closest to the operator, outside the patient) where a physician's hand H1 can guide the catheter 20. As seen in the monitor image 28 of FIG. 4, the distal end 12 of the catheter 20 may be bent at an angle.

The catheter 20 can be coupled to a cryogen source, such as a tube extending near the bottom of a Dewar flask 32 filled with liquid nitrogen or other liquefied gas LG. As shown in FIG. 4, the Dewar flask 32 is closed and the interior space is pressurized with a small air pump 34, which may alternatively be mounted in the container lid or elsewhere.

Alternatively, the apparatus may comprise a pressurized container in which the internal pressure of the container drives the flow of cryogen. Such a container can have an internal pressure from about 5 psi to 450 psi or more. Further, the container can have internal pressures of from about 20 psi to about 200 psi. The container can be a sealed canister connected to the cryospray apparatus in such a way as to permit the flow of liquefied gas from the canister without entirely releasing the pressure and allowing the cryogen to evaporate. The apparatus may include pressure step down valves or other mechanisms that reduce the pressure of the cryogen exiting the canister in order to allow the cryogen to exit the catheter at low pressure.

As used in the present specification, “gas” in the phrase “liquefied gas” means any fluid which is physiologically acceptable and which has a sufficiently low boiling point to allow the cryotherapy of the present invention. For example, such boiling point may be below about −150° C. Examples of such gases include, but are not limited to, nitrogen, as it is readily available, nitrogen oxides, oxygen, liquid air and argon. Liquefied gas may be used as a cryogen.

FIG. 4 shows schematically that the proximal end of the catheter 20 can be coupled to a tube 35, by a connector such as a standard luer lock 37, and the lower end of the tube 35 is immersed in liquid nitrogen LG while the interior is pressurized by a free-running pressure pump 34 through a tube 38. A pressure gauge 40, or alternatively a safety valve with a preset opening pressure (not shown) may be included. The pressure is selected so as to permit adequate spray from the distal end of the catheter 20. The interior of the Dewar flask 32 is vented through a vent tube 42 which can be opened and closed by a valve operated by the physician's hand H2. FIG. 4 shows the thumb obstructing the end of the vent tube 42. When the vent is closed, pressure builds up in the Dewar flask 32 and the liquefied gas is pumped through the tube 35 to catheter 20.

While the valve is shown as a simple thumb-valve in FIG. 4, it will be understood that such a valve could be a mechanical valve or an electromechanical valve, which can be controlled by a trigger mechanism, or the like, as could be readily envisioned and constructed by those of ordinary skill in the art. In an embodiment, an electrically operated solenoid valve is employed in delivering the liquefied gas to the catheter. Of course, the solenoid is specifically adapted to function properly at cryogenic temperatures.

The vent tube 42 can be left open until the physician has positioned the catheter near the respiratory tissue, as guided by the hand H1 and confirmed by viewing the monitor 28. The vent 42 is then closed and liquefied gas is pushed into the proximal end of the catheter 20 at the luer lock 37.

The apparatus shown in FIG. 3 can also be used with the methods of the present invention and is more fully described in U.S. Pat. No. 7,025,762 to Johnston et al, which is hereby incorporated by reference. Other apparatus capable of delivering liquid cryogen to a catheter, particularly low temperature, low pressure cryogen, may also be employed.

As the liquid gas moves through the catheter 20, it can start to boil and cool gas rushes ahead to emerge from the distal end or catheter tip. The boiling point of nitrogen is about −196° C. Thus, when nitrogen is used as the cryogen, low pressure liquid moving through the catheter can be less than −150° C. The amount of boiling in the catheter 20 depends on the mass and thermal capacity of the catheter. Since the catheter is of small diameter and mass, the amount of boiling can be small. After the catheter is cooled to a low temperature, and becomes filled with liquefied gas, the liquefied gas reaches the distal end of the catheter 20 near the distal end of bronchoscope 12 and begins to spray out of the catheter onto the appropriate target tissue.

In some methods, liquid cryogen is not sprayed directly upon a target tissue. Instead, the cryogen is delivered through the distal end of the catheter at a rate such that the cryogen undergoes liquid to gas phase transition before coming into contact with the target tissue. In effect, cryogen is delivered to a site of treatment as a cold gas. The cold gas causes a reduction in the ambient temperature of the region around the distal end of the catheter. As used herein, “isotherm” indicates a region of reduced ambient temperature. Thus, delivery of cryogen can be used to reduce the ambient temperature at a site to be treated. The temperature of the isotherm can be maintained at any desired value by increasing (to reduce temperature) or decreasing (to increase temperature) the rate at which cryogen is delivered through the catheter and exits the distal end of the catheter. The catheter and/or the guiding device may be equipped with a temperature sensor in order to monitor the temperature of an isotherm. Optionally, the data from the temperature sensor can be displayed on the control panel. In some embodiments, the data from the temperature sensor is used to control a valve (for example, a solenoid valve as discussed above) that controls the rate of flow of cryogen through the catheter. In such embodiments, the desired temperature of the isotherm may be programmed into the controller and the valve controlled by a feedback loop in order to maintain the desired temperature.

It is to be noted that the apparatus may be able to initiate a response in and/or freeze the tissue sufficiently without actual liquefied gas being sprayed from the catheter, and that a spray of liquid may not be needed if the very cold gas (for example, nitrogen gas at less than 0° C.) can accomplish the task of freezing the targeted tissue. Thus, an isotherm of sufficiently low temperature can be created and maintained in contact or not in contact (e.g., in proximity) with a target tissue for a period of time sufficient to result in initiating a response in the target tissue.

Freezing is apparent to the physician by the frozen tissue acquiring a white color (cryofrost), due to surface frost (visible on the monitor 28 in FIG. 4); the white color indicates respiratory tissue freezing sufficient to destroy the diseased tissue. The physician manipulates the bronchoscope 10, vent 42, and/or catheter 20 to initiate a response in and/or freeze all of the targeted tissue. Once the operation is complete, the endoscope 10 with catheter are withdrawn.

The depth of cryofrost can be controlled in three ways. First by the duration of the spray. The second, by the number of freeze/thaw cycles applied. Third, by the amount of area covered by the spray. The depth ranges of the present invention can range from superficial (epithelium) to transmural (to adventitia and beyond into the lung tissue).

Following cryofrost, the cells of the treated tissue are damaged or dying. As the treated site heals, the dead cells are removed by immune cells. Over time, healthy cells grow in their place to repair the damage and replace injured tissue.

Because the invention uses liquid spray via a catheter 20 rather than contact with a cold solid probe, there is little risk of a cold apparatus adhering to and tearing the tissue. Even if contact is made between the catheter and the tissue, the plastic material of the catheter, such as polyimide, is in little risk of sticking to the tissue because of its low thermal conductivity and specific heat. Furthermore, the catheter need not touch the tissue according to many embodiments.

In embodiments that involve spraying liquid cryogen directly onto tissue, the cooling rate (rate of heat removal) is much higher than with a solid, contact probe because the sprayed liquefied gas evaporates directly on the target tissue to be frozen, which absorbs much of the heat of vaporization. The rate of re-warming is also high, since the applied liquid boils away almost instantly. No cold liquid or solid ultimately remains in contact with the tissue, and the depth of freezing can be minimal or maximal if desired.

Cryoprobes have long been used in cryotherapy, e.g., in the airways to treat a variety of different processes such as endobronchial malignancies, etc., and studies have suggested that bronchoscopic cryotherapy may offer substantial palliation of dyspnoea, stridor, and haemoptysis in patients who have failed to respond to other treatments. Cryotherapy using cryoprobes entails contacting a target tissue with the cryoprobe thereby freezing the tissue. However, the effect of the low-pressure, physician controlled liquid nitrogen treatment in the airways has not been studied in humans.

Bronchoscopic cryotherapy employing cryoprobes has been used in endobronchial malignancies, and studies have suggested that bronchoscopic cryotherapy may offer substantial palliation of dyspnoea, stridor, and haemoptysis in patients who have failed to respond to other treatments. This method, although effective, is limited both by the surface area of the probe which is quite small, and thus is not ideal for large treatment areas and to a certain extent, incomplete tissue injury resulting from the disruption of tissue in the warmer areas of the isotherm. In fact, earlier trials of cryoablation with cryoprobes have shown these limitations, particularly when used for tumors larger than 15 mm. Residual invasive carcinoma or undetected in situ carcinoma can be left owing to incomplete freezing. Additionally, this is a touch method of treatment, which mandates the targeted treatment area come in contact with the frozen probe, and then an adequate thaw time must ensue before the probe can be removed from the tissue without ripping off the outermost layers.

Since freezing is accomplished by boiling liquefied gas (e.g., nitrogen), large volumes of this gas can be generated. It has been shown by numerous animal experiments that when used in the airways excess nitrogen gas spontaneously vents through the mouth due to the pressure differential between the lungs (chest cavity) and the room atmospheric pressure. In the same animal experiments it was also demonstrated through the use of constant pulsoximetry that the animal was fully oxygenated and suffered no degree of hypoxia from the nitrogen gas in the lungs.

However, gas can also be provided with a mechanism to escape in order to minimize the chance of pressure-related injury. Such venting mechanism may be especially useful for use with laparoscopic cryosurgical procedures or in other situations where spontaneous ventilation is insufficient. The local pressure can be higher than atmospheric because the gas can encounter resistance flowing out of inflamed respiratory airways or sites accessed by laparoscopic techniques. Thus, there is the possibility of nitrogen gas entering (or remaining, if the treatment site is within a lung) the lungs L. There can be provided several alternative methods for facilitating evacuation of gas from the respiratory tract.

First, the lungs may be suctioned with a separate tube 41, for example, a suction tube 41 as seen in FIGS. 3 and 4, which may run outside of and adjacent to the endoscope 10. Suction may be provided by a suction pump 45 or other conventional means for suction.

FIG. 2B shows a catheter tip fastened on the end of the catheter 20 and adapted to spray liquefied gas through one or more holes 49 between the surface and an interior space fed by the catheter 20. When a lateral hole is provided in the wall of the catheter, the distal end of the catheter can be closed so that cryogen is directed laterally. The length of the catheter tip and size and shape of the spray holes can be chosen so that the entire area of the targeted tissue is frozen at once without the need for manipulating the bronchoscope or catheter to initiate a response in and/or freeze the targeted area in sequential increments. The catheter tip may be of rigid material such as metal or stiff plastic. Alternatively, the entire endoscope and/or catheter may be moved up or down the respiratory tract or respiratory airway to ensure that the entire targeted area is sprayed.

FIGS. 2A, 2B, and 2C also show the distal end 12 of the bronchoscope 10 including a camera lens 14, illuminating light 16, biopsy channel or lumen 18 with the catheter 20 therein, and an additional lumen 22. The bronchoscope shown in FIG. 2 is a conventional therapeutic bronchoscope. A diagnostic bronchoscope would lack extra lumen 22.

The catheter will have one or more openings 49, whereby cryogen spray exits the catheter and contacts the tissue. The openings may be configured in such as way as to allow the cryogen to spray in a substantially perpendicular direction. The end of the catheter 20 may also be cut at an angle to deflect the spray to one side. Alternatively, FIG. 2C shows an optional cone-shaped structure 110 disposed around the opening in the catheter to direct the spray to the target tissue.

It is also contemplated that the cryospray may be supplemented with and/or used in conjunction with one or more additives. For example, cryospray may be used as a means of delivering therapeutic agents to the target tissues. Such additives may be mixed with the liquid nitrogen or other cryogen and simultaneously sprayed onto target tissue, or may be delivered (e.g., sprayed) separately from the cryogen before, during or after cryotherapy. Any suitable medium may be used to spray additives, for example, gases or liquids, which may be at the same temperature or at a higher or lower temperature than the target tissue. Non-limiting examples of contemplated additives include organic chemicals, agents, or compound formulations, inorganic chemicals or agents, gene therapy agents including but are not limited to, viruses, lipids, other transfection agents or naked circularized or linear DNA, dyes or indicators, either organic or inorganic, gels, liquids, solids, gases and crystals, glues, pharmaceuticals, prodrugs, aerosols, blood, plasma, tissue or other biological products, solvents (covered under chemicals), polymers, plasticizers, and absorbable, expandable materials, nano-technology, robotics, and/or magnetized material/products. In some aspects of the invention, oxygen can also be used as a therapeutic agent. Further, diagnostic agents including, but are not limited to, radiolabeled substances, haptens, priming agents, imaging agents, fluorescent agents, magnetic marker materials, contrast agents such as X-ray, ultrasound and MRI contrast enhancing agent, can be supplemented with cryospray.

Examples of diagnostic or therapeutic agents that can be delivered are pharmaceutically acceptable salt or dosage form of an antimicrobial agent (e.g., antibiotic, antiviral, anti-parasitic, antifungal, etc.), an anesthetic agent with or without a vasoconstriction agents (e.g. Xylocalne with or without Epinephrine, Tetracaine with or without epinephrine, etc.), an analgesic agent, a corticosteroid or other anti-inflammatory (e.g., an NSAID), a decongestant (e.g., vasoconstrictor), a mucous thinning agent (e.g., an expectorant or mucolytic), an agent that prevents of modifies an allergic response (e.g., an antihistamine, cytokine inhibitor, leucotriene inhibitor, IgE inhibitor, immunomodulator), an allergen or another substance that causes secretion of mucous by tissues, hemostatic agents to stop bleeding, anti-proliferative agents, cytotoxic agents e.g. alcohol, biological agents such as protein molecules, stem cells, genes or gene therapy preparations, viral vectors carrying DNA, proteins or mRNA coding for important therapeutic functions or substances etc.

Some nonlimiting examples of antimicrobial agents that may be used in this invention include acyclovir, amantadine, aminoglycosides (e.g., amikacin, gentamicin and tobramycin), amoxicillin, amoxicillin/clavulanate, amphotericin B, ampicillin, ampicillin/sulbactam, atovaquone, azithromycin, cefazolin, cefepime, cefotaxime, cefotetan, cefpodoxime, ceftazidime, ceftizoxime, ceftriaxone, cefuroxime, cefuroxime axetil, cephalexin, chloramphenicol, clotrimazole, ciprofloxacin, clarithromycin, clindamycin, dapsone, dicloxacillin, doxycycline, erythromycin, fluconazole, foscarnet, ganciclovir, atifloxacin, imipenem/cilastatin, isoniazid, itraconazole, ketoconazole, metronidazole, nafcillin, nafcillin, nystatin, penicillin, penicillin G, pentamidine, piperacillin/tazobactam, rifampin, quinupristin-dalfopristin, ticarcillin/clavulanate, trimethoprim/sulfamethoxazole, valacyclovir, vancomycin, mafenide, silver sulfadiazine, mupirocin (e.g., Bactroban Nasal®, Glaxo SmithKline, Research Triangle Park, N.C.), nystatin, triamcinolone/nystatin, clotrimazole/betamethasone, clotrimazole, ketoconazole, butoconazole, miconazole, tioconazole, detergent-like chemicals that disrupt or disable microbes (e.g., nonoxynol-9, octoxynol-9, benzalkonium chloride, menfegol, and N-docasanol); chemicals that block microbial attachment to target cells and/or inhibits entry of infectious pathogens (e.g., sulphated and sulphonated polymers such as PC-515 (carrageenan), Pro-2000, and Dextrin 2 Sulphate); antiretroviral agents (e.g., PMPA gel) that prevent retroviruses from replicating in the cells; genetically engineered or naturally occurring antibodies that combat pathogens such as anti-viral antibodies genetically engineered from plants known as “plantibodies;” agents which change the condition of the tissue to make it hostile to the pathogen (such as substances which alter mucosal pH (e.g., Buffer Gel and Acidform), non-pathogenic or “friendly” microbes that cause the production of hydrogen peroxide or other substances that kill or inhibit the growth of pathogenic microbes (e.g., lactobacillus); antimicrobial proteins or peptides such as those described in U.S. Pat. No. 6,716,813 (Lin et al.) which is expressly incorporated herein by reference or antimicrobial metals (e.g., colloidal silver).

Additionally or alternatively, in some applications where it is desired to treat or prevent inflammation the substances delivered in this invention may include various steroids or other anti-inflammatory agents (e.g., nonsteroidal anti-inflammatory agents or NSAIDS), analgesic agents or antipyretic agents. For example, corticosteroids that have previously administered by intranasal administration may be used, such as beclomethasone (Vancenase® or Beconase®), flunisolide (Nasalide®), fluticasone proprionate (Flonase®), triamcinolone acetonide (Nasacort®), budesonide (Rhinocort Aqua®), loterednol etabonate (Locort) and mometasone (Nasonex®). Other salt forms of the aforementioned corticosteroids may also be used. Also, other non-limiting examples of steroids that may be useable in the present invention include but are not limited to aclometasone, desonide, hydrocortisone, betamethasone, clocortolone, desoximetasone, fluocinolone, flurandrenolide, mometasone, prednicarbate; amcinonide, desoximetasone, diflorasone, fluocinolone, fluocinonide, halcinonide, clobetasol, augmented betamethasone, diflorasone, halobetasol, prednisone, dexamethasone and methylprednisolone. Other anti-inflammatory, analgesic or antipyretic agents that may be used include the nonselective COX inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and Selective COX-2 Inhibitors (e.g., diaryl-substituted furanones such as rofecoxib; diaryl-substituted pyrazoles such as celecoxib; indole acetic acids such as etodolac and sulfonanilides such as nimesulide).

Additionally or alternatively, in some applications, such as those where it is desired to treat or prevent an allergic or immune response and/or cellular proliferation, the substances delivered in this invention may include a) various cytokine inhibitors such as humanized anti-cytokine antibodies, anti-cytokine receptor antibodies, recombinant (new cell resulting from genetic recombination) antagonists, or soluble receptors; b) various leucotriene modifiers such as zafirlukast, montelukast and zileuton; c) immunoglobulin E (IgE) inhibitors such as Omalizumab (an anti-IgE monoclonal antibody formerly called rhu Mab-E25) and secretory leukocyte protease inhibitor) and d) SYK Kinase inhibitors such as an agent designated as “R-112” manufactured by Rigel Pharmaceuticals, Inc, or South San Francisco, Calif.

Additionally or alternatively, in some applications, such as those where it is desired to shrink mucosal tissue, cause decongestion or effect hemostasis, the substances delivered in this invention may include various vasoconstrictors for decongestant and or hemostatic purposes including, but are not limited to, pseudoephedrine, xylometazoline, oxymetazoline, phenylephrine, epinephrine, etc.

Additionally or alternatively, in some applications, such as those where it is desired to facilitate the flow of mucous, the substances delivered in this invention may include various mucolytics or other agents that modify the viscosity or consistency of mucous or mucoid secretions, including, but are not limited to, acetylcysteine (Mucomyst™, Mucosil™) and guaifenesin.

Additionally or alternatively, in some applications such as those where it is desired to prevent or deter histamine release, the substances delivered in this invention may include various mast cell stabilizers or drugs which prevent the release of histamine such as cromolyn (e.g., Nasal Chrom®) and nedocromil.

Additionally or alternatively, in some applications such as those where it is desired to prevent or inhibit the effect of histamine, the substances delivered in this invention may include various antihistamines such as azelastine (e.g., Astylin®), diphenhydramine, loratidine, etc.

Additionally or alternatively, in some applications such as those wherein it is desired to treat a tumor or cancerous lesion, the substances delivered in this invention may include antitumor agents (e.g., cancer chemotherapeutic agents, biological response modifiers, vascularization inhibitors, hormone receptor blockers, or other agents that destroy or inhibit neoplasia or tumorigenesis) such as; alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, triazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000) which is expressly incorporated herein by reference, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucii, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol, taxotere, analogs/congeners and derivatives of such compounds as well as other antitumor agents not listed here.

Additionally or alternatively, in some applications such as those where it is desired to grow new cells or to modify existing cells, the substances delivered in this invention may include cells (mucosal cells, fibroblasts, stem cells or genetically engineered cells) as well as genes and gene delivery vehicles like plasmids, adenoviral vectors or naked DNA, mRNA, etc. injected with genes that code for anti-inflammatory substances, etc., and, as mentioned above, osteoclasts that modify or soften bone when so desired, cells that participate in or effect mucogenesis, ciliagenesis or chondrogenesis etc.

In one embodiment of the invention comprises delivering therapeutic agents without a cryogen (for example a non-cryogenic gas), including, but are not limited to, oxygen, room air and CO₂, wherein the lesion and/or tissue comprising the lesion to be treated is not frozen upon the contact of non-cryogenic gas.

In some embodiments, the method comprises treating a target tissue, for example, a lesion and/or tissue comprising the lesion to be treated, contacting with a non-cryogenic gas for a period of time sufficient to initiate a response in and/or without freezing the lesion and/or tissue comprising the lesion. Alternatively, the lesion and/or tissue comprising the lesion to be treated may be in proximity to an isotherm having a temperature above the freezing point of the tissue for a period of time sufficient to initiate a response in and/or without freezing the lesion and/or tissue comprising the lesion.

In some embodiments, the method comprises delivering the therapeutic or diagnostic agents prior to contacting the tissue with cryogen or non-cryogenic gas, or using the cryogen or non-cryogenic gas to create an isotherm in proximity to the tissue.

In some embodiments, the method comprises delivering the therapeutic or diagnostic agents at the same time as contacting the tissue with cryogen or non-cryogenic gas, or using the cryogen or non-cryogenic gas to create an isotherm in proximity to the tissue.

In some embodiments, the method comprises delivering the therapeutic or diagnostic agents after contacting the tissue with cryogen or non-cryogenic gas, or using the cryogen or non-cryogenic gas to create an isotherm in proximity to the tissue.

In some embodiments, the method comprises mixing the therapeutic or diagnostic agents with the cryogen or non-cryogenic gas prior to contacting the tissue with cryogen or non-cryogenic gas, or using the cryogen or non-cryogenic gas to create an isotherm in proximity to the tissue.

While not wishing to be bound by any particular theory, it is contemplated that delivery of an additive with cryotherapy will facilitate cellular uptake of the additive, especially by cryotreated tissue. In vitro studies investigating the delivery of chemotherapeutic agents to frozen cells demonstrated that cold increases cellular permeability and, thereby, susceptibility to a chemotherapeutic agent that does not otherwise enter cells efficiently. Mir, LM and Rubinsky, B. (2002) Treatment of cancer with cryochemotherapy. Brit J Canc 86, 1658-1660. It is, therefore, contemplated that cryospray-induced cellular permeability may preferentially facilitate the uptake of cryospray additives into treated cells rather than non-target cells.

It is also contemplated that cells that are stimulated to grow and replicate in response to cryotherapy would rapidly assimilate biomaterials from the immediate environment. Thus, cryotherapy may make these cells less selective as to the materials they incorporate and more likely to assimilate cryospray additives. Further, when cells are immediately killed by cryofrost or sent into apoptosis following exposure, an immune response can be generated. The immune response can include a cytotoxic T cell response, a humoral response or an innate response. The immune response can involve the production of cytokines, chemokines or other signaling molecules and can involve an inflammatory response. Such mechanisms may modulate the bioavailability or cellular uptake of an additive or the metabolism of a prodrug into its active form.

If gene therapy is used, delivery vectors for gene therapy may include any suitable delivery vector known in the art, such as viruses, liposomes, nanoparticles or naked DNA.

Adenoviruses carrying deletions have been proposed as suitable vehicles for genetic information. Adenoviruses are non-enveloped DNA viruses. Gene-transfer vectors derived from adenoviruses (so-called “adenoviral vectors”) have a number of features that make them particularly useful for gene transfer for such purposes. For example, the biology of the adenovirus has been characterized in detail, the adenovirus is not associated with severe human pathology, the adenovirus is extremely efficient in introducing its DNA into the host cell, the adenovirus can infect a wide variety of cells and has a broad host-range, the adenovirus can be produced in large quantities with relative ease, and the adenovirus can be rendered replication defective by deletions in the early-region 1 (“E1”) of the viral genome.

Non-integrating viruses, such as a cytoplasmic virus, may also be a suitable vector for delivery of genetic material. The genetic material carried by these vectors will thus not be present in the nucleus of the target cell, unless specifically desired. The vector may have a low replicative efficiency in the target cell.

Non-lytic viruses, those that will not kill most target cells in the host animal or a tissue culture in a short period of time during which the viable infected cells will be expressing the gene product, may also be used. For example, it may not kill more than about 25% of the target cells it is being used in within 48 hours, 72 hours or 96 hours. Further, it may not kill more than about 10% of the target cells in the host animal or tissue culture it is used in within 48 hours, 72 hours or 96 hours. In addition, such a transformed target cell population may be expressing the delivered gene product for a period of 1 to 2 weeks after initial infection. This can readily be determined by assaying samples of the target cell for viability, e.g., by staining with trypan blue, and gene expression, e.g., measuring protein production with ELISA.

The term “short-term” delivery system described herein is can be directed to the use of vector systems that although capable of expressing the desired genetic material for at least about 1 week will result in the transient expression of the gene product. The expression can be for less than about 2 months or less than about 1 month. In addition, by using an avirulent virus for the selected animal host the virus will not cause disease in the host. If any adverse effects are observed, such effects can be further curtailed as described below. Moreover, the delivery system described herein is capable of “controlled release” of a desired protein or other gene product by continuously expressing specific amounts of the protein over a given period of time.

Suitable non-integrating viruses are cytoplasmic viruses. These include both DNA and RNA viruses. DNA viruses includes poxviruses such as suipox (e.g. swine pox) capripox, leporipox, avipox (e.g. fowl pox, canary pox) and orthopox (e.g. ectomelia, rabbit pox). Other DNA viruses include iridoviruses such as various insect and frog viruses.

RNA viruses include picornaviruses, caliciviruses, togaviruses, rhaboviruses and coronaviruses. Picornaviruses include enterovirus, cardiovirus, rhinovirus, apthovirus, and hepatitis A. Calicivirus include vesicular exanthema virus of swine, dogs or mink, feline calicivirus and caliciviruses of calves, swine, dogs, fowl and chimps. Togaviruses include bovine viral diarrhea virus, hog cholera, and border disease of sheep. Rhabdoviruses include vesiculoviruses such as vesicular stomatitis virus and Lyssaviruses such as rabies. Coronaviruses include infectious bronchitis virus of fowl, transmissible gastroenteritis virus of swine, hemagglutinin encephalyomyelitis virus of swine, turkey, bluecomb virus, calf coronavirus and feline infectious peritonitis virus.

DNA viruses may also be used as vectors. For example, pox viruses are well known cytoplasmic viruses. Thus, genetic material expressed by such viral vectors typically remain in the cytoplasm and do not have the potential for inadvertent integration of the genetic material carried into host cell genes, unless specific steps are taken such as described above. Furthermore, because these vectors have a large genome, they can readily be used to deliver a wide range of genetic material including multiple genes (i.e., act as a multivalent vector).

The viral vectors may be oncolytic viral vectors. Oncolytic viral vectors are viral vectors which selectively replicate in tumor cells and destroy the cells in which they replicate, but do not replicate to any significant degree, in non-tumor cells. For example, oncolytic adenoviral vector may have a tissue-specific transcriptional regulatory sequence is operably linked to said gene essential for replication as described above. Alternatively, oncolytic adenoviral particles may include a mutation in a gene essential for adenoviral replication, such as the E1a or E1b genes. Such mutations may render adenoviral replication specific for tumor tissue, e.g. if the cells of said tissue have a defect in the p53 or Rb pathways. Oncolytic adenoviral vectors may or may not include a heterologous gene in addition to the adenoviral elements necessary for replication.

In a further embodiment, the present invention provides vector constructs which include a therapeutic gene. A therapeutic gene can be one that exerts its effect at the level of RNA or protein. For instance, a protein encoded by a therapeutic gene can be employed in the treatment of an inherited disease, e.g., the use of a cDNA encoding the cystic fibrosis transmembrane conductance regulator in the treatment of cystic fibrosis. The protein encoded by the therapeutic gene can exert its therapeutic effect by causing cell death. For instance, expression of the protein, itself, can lead to cell death, as with expression of diphtheria toxin A, or the expression of the protein can render cells selectively sensitive to certain drugs, e.g., expression of the Herpes simplex (HSV) thymidine kinase gene renders cells sensitive to antiviral compounds, such as acyclovir, gancyclovir and FIAU (1-(2-deoxy-2-fluoro-.beta.-D-arabinofuranosil)-5-iodouracil). Alternatively, the therapeutic gene can exert its effect at the level of RNA, for instance, by encoding an antisense message or ribozyme, a protein that affects splicing or 3′ processing (e.g., polyadenylation), or a protein that affects the level of expression of another gene within the cell, e.g. by mediating an altered rate of mRNA accumulation, an alteration of mRNA transport, and/or a change in post-transcriptional regulation.

Tumor suppressor genes are genes that, in their wild-type alleles, express proteins that suppress abnormal cellular proliferation and may also be delivered or upregulated as part of cryotherapy. When the gene coding for a tumor suppressor protein is mutated or deleted, the resulting mutant protein or the complete lack of tumor suppressor protein expression may fail to correctly regulate cellular proliferation, and abnormal cellular proliferation may take place, particularly if there is already existing damage to the cellular regulatory mechanism. A number of well-studied human tumors and tumor cell lines have been shown to have missing or nonfunctional tumor suppressor genes. Examples of tumor suppression genes include, but are not limited to, the retinoblastoma susceptibility gene or RB gene, the p53 gene, the deleted in colon carcinoma (DCC) gene and the neurofibromatosis type 1 (NF-1) tumor suppressor gene (Weinberg, R. A. Science, 1991, 254:1138-1146). Loss of function or inactivation of tumor suppressor genes may play a central role in the initiation and/or progression of a significant number of human cancers.

For human patients, the therapeutic gene will generally be of human origin although genes of closely related species that exhibit high homology and biologically identical or equivalent function in humans may be used if the gene does not produce an adverse immune reaction in the recipient. As used herein, the term “high homology” refers to genes that have 85%, 90%, 95% or 99% identical base pairs. A therapeutically effective amount of a nucleic acid sequence or a therapeutic gene is an amount effective at dosages and for a period of time necessary to achieve the desired result. This amount may vary according to various factors, including, but are not limited to, sex, age, weight of a subject, and the like.

The DNA sequence encoding at least one therapeutic gene is under the control of a suitable promoter. Suitable promoters which may be employed include, but are not limited to, adenoviral promoters, such as the adenoviral major late promoter; or hetorologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter. In one embodiment, the promoter of the invention is an E2F-responsive promoter, in particular the E2F-1 promoter. In one embodiment of this invention, the E2F promoter is operatively linked to the E1a gene.

In addition to the E2F promoter, use of the following tumor selective promoters are contemplated: osteocalcin, L-plastin, CEA, AVP, c-myc, telomerase, skp-2, psma, cyclin A, and cdc25 promoters. It is to be understood, however, that the scope of the present invention is not to be limited to specific foreign genes or promoters. The selection of a particular promoter and/or enhancer depends on what cell type is to be used to express the protein of interest. Some eukaryotic promoters and enhancers have a broad host range while others are functional in a limited subset of cell types.

The liposome compositions can provide highly efficient delivery of biologically active agents to cells. Liposome vesicles can be prepared from a mixture of a cationic lipopolyamine and a neutral lipid and form a bi- or multilamellar membrane structure (referred to herein as “DLS-liposomes”). For example, one may use a spermine-5-carboxy-glycinedioctadecylamide (referred to herein as “DOGS”) as the cationic lipopolyamine and dioleylphosphatidyl ethanolamine (referred to herein as “DOPE”) as the neutral lipid. Other liposome compositions can also be used. Use of such liposomal vehicles make possible high transfection efficiency of biologically active materials into cells.

The presence of at least one neutral lipid in combination with at least one cationic lipopolyamine makes possible the formation of liposomes after hydration. Liposomes may be prepared by mixing together each of a cationic lipopolyamine and a neutral lipid in a molar ratio ranging from, for example, a ratio of 0.02:1 to a ratio of 2:1; evaporating the mixture to dryness; and rehydrating. In order to introduce a biologically active agent into the liposomes, such agent can be added prior to or after rehydration of the dried film.

Nucleic acids may be associated with the liposomes. This association may be accomplished in at least two ways: (1) complex formation between the cationic liposome vesicle and negatively charged polyaminon, such as nucleic acid or (2) encapsulation in the cationic liposome vesicle. Such a formulation may have applications for treating subjects via effective delivery of oligonucleotides or gene-expressing nucleic acid vectors (e.g. plasmids or viral vectors) into cells. Therefore, such a method of drug delivery is useful for the transport of nucleic acid based therapeutics.

It is also contemplated that cryotherapy may be utilized to manipulate immune system responses in the lungs. While not wishing to be bound by any particular theory, it is contemplated that cells critically damaged by cryospray will initiate their apoptotic machinery. These dead and dying cells may recruit immune effecter cells, such as macrophages or other phagocytes and T helper cells, to the treated site.

By taking advantage of this mechanism, it is contemplated that cryotherapy may be used to initiate a targeted immune response in respiratory tissue for the treatment of a lung disease. Recruiting immune cells to a site of pathology may increase the likelihood of encounter and, thus, allow the immune system to recognize a tumor cell, pathogen, or other cells that may otherwise evade the normal innate or adaptive immune system responses. Such methods may be used to treat lung cancer, lung infections, or other conditions that may benefit from an increased or targeted immune response. An inflammatory response associated with same may also beneficially effect the desired therapy. For example, inflamed tissue can be more permeable to therapeutic agents than non-inflamed tissue.

It is also contemplated that cryotherapy may be used to suppress inflammation as well as to induce a systemic immune and antimetastatic response. Cryotherapy is frequently used to treat and alleviate inflammation of other parts of the body as well as to induce a systemic immune and antimetastatic response, such as by application of ice packs to injured muscle tissue. While not wishing to be bound by any particular theory, it is contemplated that cryospray therapy may be used to cool target lung tissue without developing cryofrost and cellular damage or death. Alternatively, more intense cryotherapy may be used to initiate a response in and/or freeze and kill nerve endings that are sending pain signals, thereby inducing an analgesic effect. Such cryo treatment may alleviate swelling, heat, and pain of respiratory tissue and pleural spaces caused by inflammation.

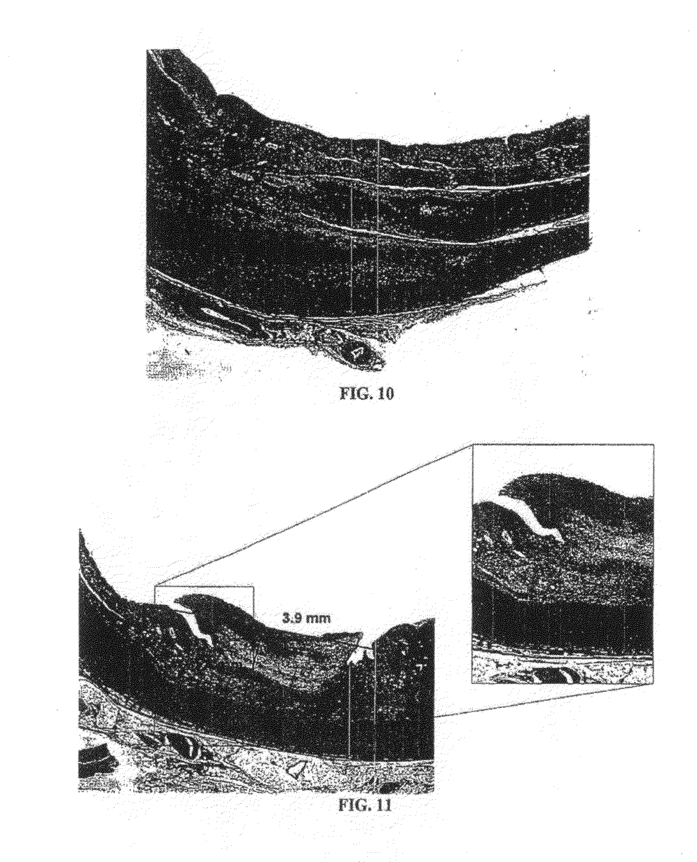

In a further contemplated embodiment, it is envisioned that cryotherapy may be used to stimulate chondrogenesis. Cartilage, for example, of the bronchi or bronchioles that has been damaged due to physical injury, chronic inflammation, or any other cause may be treated with cryospray. The regeneration of cartilage has been observed after cryotherapy. FIG. 10 shows chondrogenesis after 28 days of healing in swine treated with cryotherapy. Cartilage in portions of the body other than the thoracic cavity may also be treated using cryotherapy. In one embodiment, cartilage in a joint may be treated with cryotherapy.

It is also contemplated that cryotherapy may have useful application in tissue transplantation. For example, early studies involving the transplant of cadaverous aorta tissue into the airway of a recipient sheep suggest that cryotherapy may be helpful in generating immune neutral tissue transplant and stimulating chondrogenesis and the growth of ciliated epithelium in the aorta tissue. While not wishing to be bound by any particular theory, it is believed that cryotherapy performed on transplanted tissue or surrounding tissue could stimulate growth of epithelial or other tissues, intercellular signaling and/or response to signals that may promote the generation of new tissues or the expression of a desired phenotype in the transplanted tissues. In some embodiments, a site into which tissue is to be transplanted is first treated with cryogen. The treatment may result in freezing of the target site. After treatment with cryogen, the tissue to be transplanted may be attached to the treated site. A period of time may be allowed to elapse between treatment and attachment.

When performing cryotherapy procedures, the cryogen spray can be conducted in such a manner as to allow constant direct visualization by the physician of the targeted tissue treatment as it occurs. If the temperature of the lens at the proximal end of the bronchoscope (if used) drops precipitously at the start of the liquefied gas spray, the moist air of the respiratory environment or of the air of the catheter which has been blown out ahead of the liquefied gas flow can condense on the lens, thereby obscuring the physician's view of the operative site. This can be substantially avoided by means of the suction pump 45 which will immediately suck out the moist air which is present prior to the arrival of the liquid spray or cold gas. However, it has been found that fogging normally clears on its own when cryotherapy is performed in the airway, thereby eliminating the need for suction in many circumstances. Because of this pumping out of the moist air as the spray commences and the replacement with extremely dry gas, substantial amounts of moisture will not form on the lens 14 during the procedure, allowing an excellent view of the operative site by the physician during the procedure.

This condensation effect is augmented by the fact that the catheter itself may not be wrapped in additional insulation. This causes the temperature of the liquefied gas exiting the catheter at the distal end to be relatively high at the beginning of the spraying operation and gradually cooling as the catheter cools. Indeed, in the tests conducted in the respiratory tracts and respiratory airways of pigs discussed below in the Examples, 10-20 seconds may be necessary before significant freezing is seen through the bronchoscope. If the catheter is substantially insulated, the interior of the catheter will cool much more quickly as it will not be picking up heat from the outside. With this insulated catheter, it is to be expected that the liquefied gas would be sprayed onto the target tissue almost immediately, causing much faster freezing and, thus, allowing less control on the part of the physician.

Another reason that the lens does not fog or frost in the present invention is that the respiratory tract or respiratory airway can be flushed out with the liquefied gas, which is extremely dry. The liquefied gas is moisture free because it is condensed out of atmospheric gases at a temperature −197° C. (when nitrogen is used), colder than the temperature at which moisture is condensed out.

The combination of relatively warm, and completely dry nitrogen gas, with or without suction, flushes moist air from the respiratory tract or respiratory airway. As the temperature of the liquefied gas entering the respiratory tract or respiratory airway falls, so does the surface temperature of the camera lens 14. Ordinarily at that time the lens 14 would be cold enough to condense moisture and fog, however, since the respiratory tract or airway is dried out (in contrast to its usual highly moist state) there is little or no moisture to condense. Thus, the lens 14 stays un-fogged and un-frosted and continues to provide a clear view of the operation. On the other hand, if the respiratory tract or airway is not vented with suction and/or the respiratory tract or airway is not preliminarily flushed with dry gas (perhaps because the catheter is insulated, lowering its heat capacity, and/or the liquefied gas delivery pressure is too high), then the lens may fog or frost and the physician cannot operate effectively for a limited time.

In order to deal with the moist air problem a suction tube 41 (FIGS. 3 and 4) can be supplied if spontaneous venting is not found to be adequate. During the cryosurgical procedure the suction tube can be inserted prior to inserting the bronchoscope 10 and catheter 20. The suction tube 41, when connected to a pump 45, can serve to evacuate moist air from the respiratory tract or airway prior to cryosurgery. With moist air removed, the television camera lens 14 is not obscured by fog and the physician can perform cryosurgery with an unobstructed view. Alternatively, if fogging occurs during cryosurgery, the suction tube and pump can be used to evacuate the respiratory tract or airway.

The composition of the catheter or the degree of insulating capacity thereof can be selected so as to allow the freezing of the targeted tissue to be slow enough to allow the physician to observe the degree of freezing and to stop the spray as soon as the surface achieves the desired whiteness of color (cryofrost). The clear observation results from the removal of the moist air and sprayed liquefied gas by the vacuum pump; in combination with the period of flushing with relatively warm liquefied gas prior to application of the spray of the liquefied gas which is caused by the relative lack of insulation of the catheter. The catheter can have a degree of insulation which permits at least five seconds to pass from the time said means for controlling is opened to the time that liquefied gas is sprayed onto the targeted tissue.

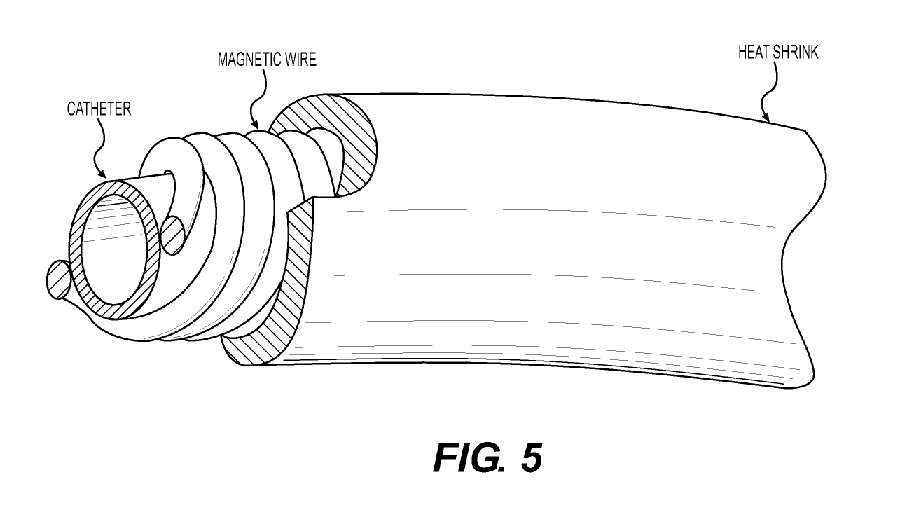

In another embodiment, the catheter used in the method of the present invention can be a heated catheter. The heated catheter can be a composite constructed of three different materials; in three different layers. The catheter itself (as the first layer) can be made of extruded polyimide. Surrounding the first layer (the catheter) can be a layer of magnetic wire wrapped around the outer diameter of the polyamide catheter. As a top or final layer, there can be supplied a thin polyester heat shrink. A heated catheter is exemplified in FIG. 5. U.S. patent application Ser. No. 10/352,266 describes additional heated catheters and associated apparatus that can be used with the methods described herein, and is hereby incorporated by reference.

The heated catheter can provide a number of advantages over a traditional catheter: Polyimide, the cryo-catheter material base, acts as a strong insulator and transports the liquid nitrogen with minimal thermal temperature loss resulting in a shorter time to achieve the clinically required cryofrost. The heating mechanism allows the catheter to be removed from the endoscope lumen immediately following the cryo-therapy. Using a traditional catheter, the catheter can freeze to the endoscope lumen during the therapy, and may not thaw for 30-40 seconds or more following the therapy. This freezing to the endoscope lumen may result in damage to the endoscope, particularly if the operator attempts to remove the catheter from the lumen before it has thawed sufficiently.

An electronic monitoring and recording system may also be used with the apparatus during cryosurgery of the respiratory system and is described in U.S. Pat. No. 7,025,762. The electronic components of the system may comprise a temperature sensor or probe and timer. Also connected to the monitoring and recording system may be a foot-pedal for actuating the solenoid and a recording console. An electric power cord can run from solenoid to control box. The electronic monitoring and recording system may record the times at which cryofrost starts and ends. Temperature in the context of time may also be recorded for the cryosurgery. This recordation allows for better data acquisition and documentation. The electronic console can be preprogrammed to be patient specific.

The components or paraphernalia required to practice the method of the present invention may be packaged and sold or otherwise provided to health-care providers in the form of a kit. The kit is can be sealed in a sterile manner for opening at the site of the procedure. The kit can include the catheter, having the spray means at one end, as well as a means for connecting the catheter to the source of liquefied gas. This means for connecting may be a simple luer connection on the opposite end of the catheter from the spray means. However, the term “means for connecting said catheter to a source of liquefied gas” is intended to include any other device or apparatus which allows the catheter to be connected to the gas source.

Certain of the components of the cryosurgical system can be conventional medical appliances. For example, the bronchoscope can be a conventional medical appliance and would not necessarily have to be supplied as part of a kit. One of the components to be supplied in a kit or sterilized package can be a combined catheter-bleeder vent. The catheter may be integrally provided with a pressure reducing bleeder vent at its proximal end as a single unit. The catheter and bleeder unit can be supplied with various modifications in the placement of the bleeder vent relative to the catheter as described in U.S. Pat. No. 7,025,762.

The unit can be attached to the gas supply tube through a luer lock connection and can be supplied to the user in a sterile package or kit. The bronchoscope may either be part of the kit or an available conventional bronchoscope may be used in conjunction with the remaining components of the kit. The kit may also optionally contain means for withdrawing gas, such as a tube and a means connectable to the tube for withdrawing gas from the tube. Such means connectable to the tube for withdrawing gas may be a vacuum pump or any other device or apparatus which will accomplish the function of withdrawing gas from the tube. The vacuum pump is optionally omitted from the kit as a source of vacuum is often found in hospital rooms or practitioner offices in which such a procedure is to take place.

The term “container” or “package” when used with respect to the kit is intended to include a container in which the components of the kit are intended to be transported together in commerce. It is not intended to comprehend an entire procedure room in which the individual components may happen to be present, an entire vehicle, a laboratory cabinet, etc.

When used in connection with a spray pattern, the term “substantially perpendicular” is not intended to limit direction of the spray to a plane at an angle of 90 degrees to the axis of the catheter, but includes any type of spray which will allow the targeted tissue of the lumen, such as the respiratory tract or airway that is coaxial to the catheter to be sprayed, near the locus of the tip of the catheter and to exclude a spray which is only substantially axial.

The phrase “means for controlling the flow of liquefied gas” is intended to encompass the simple thumb-valve illustrated in FIG. 4, as well as any other mechanical, mechano-electrical, etc., device that will accomplish the function of controlling the flow of liquefied gas from the source to the catheter. This includes any type of valve, including, for example, a trigger valve, a rotary valve, a stopcock, etc. The valve may be manually controlled, electrically driven, remotely controlled, etc. Other means for controlling the flow of liquefied gas are not excluded.

The phrase “means for withdrawing gas” is intended to include the illustrated tube 41 and vacuum pump 45, as well as any functional equivalent thereof, including the lumen of a bronchoscope used as a gas venting member, or a tube withdrawing the gas that passes through the bronchoscope, around the bronchoscope, or is placed into the area from which gas is to be withdrawn by incision. The only important function is the withdrawal of the gas from the area in question. Including, but are not limited to, a vacuum pump, any other type of pump or device which will cause the withdrawal of the gas is intended to be encompassed by this terminology. Other means for withdrawing gas are not excluded.

The phrase “means for forcing said liquefied gas” is intended to include not only the illustrated pressure pump 34 but any other device or apparatus which will force the liquefied gas from its source to the catheter. This includes use of a pre-pressurized container of liquefied gas or apparatus which causes gas to liquefy and then be directly directed to the catheter, etc. No manner of driving the liquefied gas from the source to the catheter is intended to be excluded.

Each of the steps set forth in the method claims herein are likewise intended to comprehend not only the specific acts described in the specification, but any other acts which will accomplish the function set forth in the method step. Thus, for example, the step of adjusting the catheter may be accomplished by hand or by any other technique up to and including use of a complicated remote controlled robotic adjusting apparatus. The same is true for all of the other method steps for performing specified functions.

The preliminary test results indicate that a 5 second “cryofrost” time over varying cycles was adequate to ensure the appropriate tissue destruction, and thus appropriate cellular healing of damaged tissue for many applications. “Cryofrost” is a term defined by the instance that the normally “pinkish” targeted tissue turns white (much like freezer burn). A range for the “cryofrost” time could be about 5-10 seconds to about 2 minutes or more depending on the substrate to be treated.

Due to the nature of the system, “cryofrost” may not immediately occur, but may require that the fitting and catheter system become cool so that cryogen being sprayed from the distal end of the catheter is adequately-cold to effect the cryofrost. This can require approximately 20-30 seconds from the time that the cryogen begins to flow. Of course, this time may be longer or shorter depending on the temperature of the cryogen, the length of the flow path, the materials from which the system is constructed and environmental conditions.

During animal testing the approximate temperature that cryofrost was first observed was at approximately −10° C. The temperature range for cryofrost would be approximately −10 to −90° C.

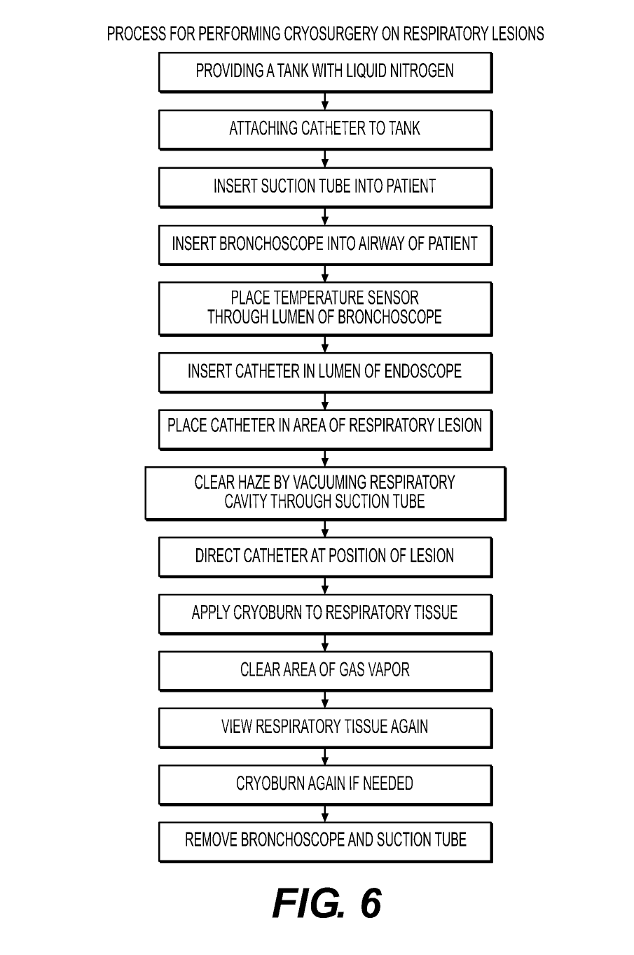

The steps for performing the respiratory tract or airway cryosurgical procedure are shown in the flow chart of FIG. 6. A cryogen source is provided. The proximal end of a suitable catheter is attached to the cryogen source so as to be in fluid communication therewith once the source is activated. If necessary, a suction tube, attached at a proximal end to a suction device can be inserted into the respiratory tract such that the distal end of the suction tube is near the target tissue or otherwise in fluid communication with the treatment space surrounding the tissue. The distal end of the suction tube can be positioned proximal to the target tissue so as not to interfere with the treatment. If suction is to be performed through a bronchoscope or not performed, the suction tube can be omitted. The bronchoscope can be inserted into the patient such that the distal end of the scope is near the target tissue and the tissue is visualized. The bronchoscope can be supplied with light and a fiberoptic visualization system or television camera. Optionally, attached to the bronchoscope will be a temperature probe to sense the temperature and report the temperature to the recording console, or a temperature sensor can be placed through a lumen of the bronchoscope. The distal end of the catheter can then be inserted through the working channel (lumen) of the bronchoscope. In the event that the distal end of the catheter includes a directional tip that does not fit in the lumen, it is possible to thread the proximal end of the catheter through the bronchoscope and connect the proximal end to the cryogen source after it has been inserted. The distal tip of the catheter can be positioned near the tissue to be treated, with the spray tip (open distal end or lateral hole) directed at the tissue. The respiratory tract or airway can be vented using the suction tube to remove moist air (if required). A cryofrost can be applied to the tissue by spraying cryogen at low pressure and low temperature. Cryogen will come from the tip of the catheter. The cryofrost treatment can last for about 30 seconds to about 2 minutes. Shorter or longer times may be appropriate depending on the size and nature of the tissue to be treated. Cryospray can be administered in a series of cycles. The tissue can be visualized between cryospray cycles or when the treatment is complete to ensure adequate cryofrost, and treatment repeated if necessary. Once the desired cryofrost has been achieved, the bronchoscope can be removed.

During the procedure, the ventilator circuit can be opened to atmosphere while still pumping pure oxygen. This is a major advantage over all burning modalities. The distal end of the ET tube in relation to the catheter if the distal end of the catheter is in the trachea. There may be no need for a ventilator according to some embodiments.

Cryotherapy may be useful in treating, preventing, or curing lung diseases such as, but are not limited to, obstructive lung and thoracic disorders, interstitial and granulomatous diseases, benign or malignant tumors or lesions and neoplastic diseases of the chest, infectious disease of the chest, pulmonary vascular diseases, pleural diseases, occupational lung disease, drug-induced lung disease, respiratory distress syndrome/bronchopulmonary dysplasia, and a variety of conditions characterized by inflammation of lung tissue, pleural tissue, chest wall tissue, as well as to induce a systemic immune and antimetastatic response.

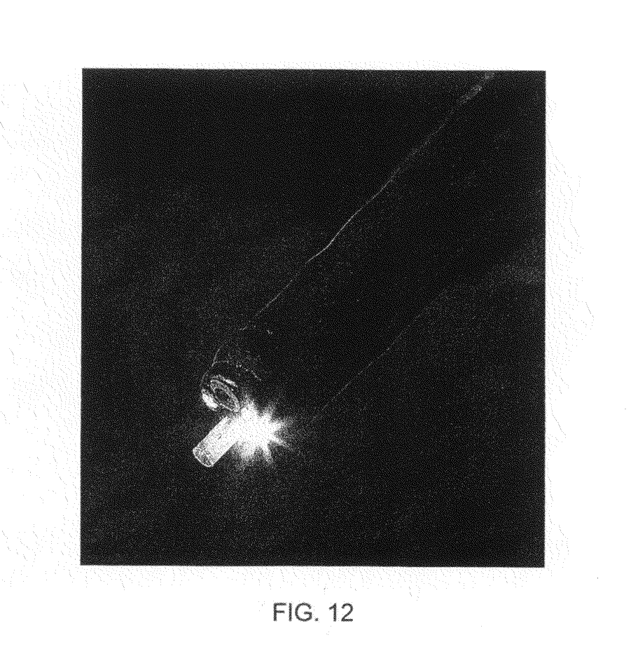

The methods of the present invention can be performed using the CryoSpray Ablation™ System (Model CC2-NAM, CSA Medical, Inc), which is a cryosurgical device intended to be used as a cryosurgical tool for the destruction of unwanted tissue. Medical Grade liquid nitrogen can be applied to unwanted tissue via the CSA™ Catheter, which is introduced through the working channel of a therapeutic bronchoscope. The system enables the physician to control the start and stop of cryogen flow and thus the duration of the cryogen spray to the selected site. Freezing techniques are monitored by direct visualization with a bronchoscope. FIG. 12 shows the catheter in close up orientation.

The CryoSpray Ablation™ System is an FDA cleared, Class II device “intended to be used as a cryosurgical tool for the destruction of unwanted tissue in the field of general surgery, specifically for endoscopic applications” (K072651). As defined by the FDA, the CSA System is a cryosurgical unit with a liquid nitrogen cooled cryocatheter and accessories used to destroy tissue during surgical procedures by applying extreme cold. This delivery of liquid nitrogen results in tissue ablation and allows for the regrowth of normal, healthy tissue. Therapeutic application of cold technology is widely used in a number of medical fields such as dermatology, gynecology, and in the treatment of esophageal disease.

The evidence from this work as well as that of this study has lead to ongoing trials for benign and malignant airway lesions. Two clinical uses of the CryoSpray System in the pleural space have lead to an efficacy study in this arena as well. The loss of smooth muscle noted in this study and in the animal studies have also lead to an investigation of usefulness of CSA therapy in Asthma, as well as chronic bronchitis and emphysema.

Embodiments of the invention are further illustrated by the following non-limiting prophetic examples showing application of cryotherapy to treat lung disorders.

Obstructive Lung And Thoracic Disorders

Chronic Obstructive Pulmonary Disease (COPD)

Chronic obstructive pulmonary disease (COPD) is a term typically referring to two lung diseases, chronic bronchitis and emphysema, that are characterized by obstruction to airflow that interferes with normal breathing. Both of these conditions frequently co-exist, hence physicians prefer the term COPD.

Chronic Bronchitis

Chronic Bronchitis is defined clinically as a persistent cough that produces sputum (phlegm), for at least three months in two consecutive years. There is no cure for chronic bronchitis. The goal of treatment is to relieve symptoms and prevent complications and exposure to irritants.

Mucus is primarily produced by secretory granules of specific dedicated mucus-producing cells, known as goblet cells. In health, goblet cells are present in large airways, becoming increasingly sparse towards the lung periphery, with few or none being found in the small airways. Submucosal glands are restricted to large airways in all species in which they occur, and in humans their density decreases with airway diameter such that glands are no longer present in small non-cartilaginous airways. In chronic respiratory disease, submucosal glands increase in size and goblet cells increase in number, appearing in the small airways via phenotypic conversion from non-goblet cells, a process termed metaplasia. The terminal and respiratory bronchioles cannot be cleared by cough and do not possess the same mucociliary clearance capacity of the larger airways. Therefore, excess mucus production at these sites can be particularly difficult to clear and is thought to contribute to occlusion of the small airways.

It is contemplated that excessive mucus production can be reduced by cryogen spray ablation of hypersecretory or abnormally localized mucus producing cells, for example, goblet cells. It is expected that mucus producing cells affected by cryofrost will die and be replaced by normal tissue that does not contribute to excessive mucus production, thereby curing or attenuating the symptoms of chronic bronchitis. Areas of excessive mucous production can be treated with cryogen

Emphysema

Emphysema is a type of chronic obstructive lung disease. It is often caused by exposure to toxic chemicals or long-term exposure to tobacco smoke and is characterized by loss of elasticity of the alveoli. When toxins, such as smoke, are breathed into the lungs, the particles are trapped and cause a localized inflammatory response. Chemicals released during the inflammatory response can damage the walls of alveoli. This leads to fewer but larger alveoli, with a decreased surface area and a decreased ability to absorb oxygen and exude carbon dioxide by diffusion. Emphysema may affect the right and left lung differently, or may be more or less severe in different lobes of a single lung. Often, the upper lobes show severe pathology.

Current treatment of emphysema includes drug therapies that temporarily aid in breathing, supplemental oxygen, and Lung volume reduction surgery (LVRS). LVRS involves the resection of a portion of a patients lung to remove the affected lobe or portion thereof. LVRS can conventionally remove approximately 20-35% of the poorly functioning, space occupying lung tissue from each lung. By reducing the lung size, the remaining lung and surrounding muscles (intercostals and diaphragm) are able to work more efficiently. Surgery, however, is invasive and not available to patients in the later stages of disease progression.

It is contemplated that cryotherapy will provide a less invasive procedure that treats diseased tissue or lobe and also potentially cures the disease. It is expected that the application of cryofrost to damaged tissue and ablation of the diseased aveoli will stimulate to regeneration of normal, healthy aveoli.

Lung volume may also be reduced using cryotherapy. In one contemplated method, cryotherapy may be used to effect a fibrotic response, thereby effecting a reduction in lung volume. This therapy would involve prolonged (greater than 10 to 20 seconds) therapy of supercooled gas. Presently, cryogen delivery cycles of 30 to 60 seconds are contemplated. Alternatively, lobectomy may be performed using more intense cryotherapy sufficient to extensively damage the lung tissue and prevent future healing. Several treatments may be administered over time to expose tissue in distal branching passages of the bronchioles to cryotherapy.

Bronchiectasis

Bronchiectasis is an abnormal stretching and enlarging of the respiratory passages caused by mucus blockage. When the body is unable to get rid of mucus, mucus becomes stuck and accumulates in the airways. The blockage and accompanying infection cause inflammation, leading to the weakening and widening of the passages. The weakened passages can become scarred and deformed, allowing more mucus and bacteria to accumulate, resulting in a cycle of infection and blocked airways.

Bronchiectasis is one of the chronic obstructive pulmonary diseases (COPD) and it can be complicated by emphysema and bronchitis. The disease is commonly misdiagnosed as asthma or pneumonia. Bronchiectasis can occur as part of a birth defect, such as primary ciliary dyskinesia or cystic fibrosis. About 50% of all cases of bronchiectasis in the U.S. result from cystic fibrosis. It can also develop after birth as a result of injury or other diseases, like tuberculosis, pneumonia and influenza.

It is contemplated that cryotherapy can be used to ablate bronchial tissues that have been scarred, damaged, or deformed and stimulate the growth of healthy tissue. Due to the chondrogenic effects of cryotherapy, Bronchiectasis patients would especially benefit from regeneration of the cartilage of collapsed or distended portions of the bronchi.

Additionally, as Bronchiectasis is often associated with other lung diseases, such as COPD or cystic fibrosis, treatment of Bronchiectasis may also be tailored to address the associated diseases. For example, in a patient affected by both Bronchiectasis and cystic fibrosis, cryospray with additional CFTR gene therapy agents may be applied to the lungs in conjunction with cryotherapy of the bronchi.

Asthma

Asthma is a chronic disease of the respiratory system in which the airway occasionally constricts, becomes inflamed, and is lined with excessive amounts of mucus, often in response to one or more triggers. The symptoms of asthma, which can range from mild to life threatening, can usually be controlled with a combination of drugs and environmental changes. Recently, surgical procedures have been designed to prevent or reduce the ability of airway smooth muscle to contract and have the potential to reduce airway responsiveness, the severity and frequency of asthma symptoms, the medications required by patients, and perhaps to improve baseline lung function.

Bronchial thermoplasty (BT) is one therapy designed to reduce the contractile ability of airway smooth muscle. BT is the delivery of radiofrequency energy to the airway wall, which heats the tissue in a controlled manner and aims to reduce smooth muscle mass. Consequently, there is decreased potential for bronchoconstriction and possibly decreased frequency and severity of asthma symptoms.

It is contemplated that cryosurgery ablating the bronchial smooth muscle tissue could provide similar benefits without the risks of excessive tissue damage associated with heat ablation techniques.

Air Way Stricture

Cryotherapy may also be helpful in creating or relieving a stricture of the airway that obstructs breathing. Tissues that contribute, or have contributed to the airway obstruction may be ablated by cryospray. After healing, it is expected that the stricture will have been destroyed and replaced by tissue that does not obstruct the airway.

Neoplastic Diseases in the Chest

It is contemplates that cryotherapy may be used for treating forms of neoplastic diseases such as, but are not limited to, Primary Lung Cancer, Mesothelioma, Carcinoid, Metastatic Disease, both solid organ and hematologic, Myeloproliferative disorders, Lymphoproliferative Disorders.

There are two major types of lung cancer. Non-small cell lung cancer is the most common. It usually spreads to different parts of the body more slowly than small cell lung cancer. Squamous cell carcinoma, adenocarcinoma, and large cell carcinoma are three types of non-small cell lung cancer. Small cell lung cancer accounts for less than 20% of all lung cancer.

The expected 5-year survival rate for all patients in whom lung cancer is diagnosed is 15.5 percent compared to 64.8 percent for colon, 89 percent for breast and 99.9 percent for prostate cancer. The 5-year survival rate is 49.3 percent for cases detected when the disease is still localized. However, only 24 percent of lung cancer cases are diagnosed at an early stage. For distant tumors the 5-year survival rate is just over 2 percent.

Mesothelioma is a rare form of cancer that involves the mesothelium, or cells that line an organ, usually the lungs, abdominal organs, and heart. The most common form of mesothelioma is pleural mesothelioma, where malignant tumors form on the pleura, the sac that lines the chest cavity and protects the lungs. Mesothelioma can be caused by asbestos exposure. Treatment for mesothelioma can be surgery to remove the tumors, chemotherapy, radiation, or a combination of the three.

Hamartoma

A hamartoma is a common benign tumor in an organ composed of tissue elements normally found at that site but that are growing in a disorganized mass. They occur in many different parts of the body and are most often asymptomatic and undetected unless seen on an image taken for another reason. Hamartomas result from an abnormal formation of normal tissue, although the underlying reasons for the abnormality are not fully understood. They grow along with, and at the same rate as, the organ from whose tissue they are made, and, unlike cancerous tumors, only rarely invade or compress surrounding structures significantly.

The most common hamartomas occur in the lungs. About 5-8% of all solitary lung tumors, about 75% of all benign lung tumors are hamartomas. They almost always arise from connective tissue and are generally formed of cartilage, fat, and connective tissue cells, although they may include many other types of cells. The great majority of them form in the connective tissue on the outside of the lungs, although about 10% form deep in the linings of the bronchi. They can be worrisome, especially if situated deep in the lung, as it is important and sometimes difficult to distinguish them from malignancies. An x-ray will often not provide definitive diagnosis, and even a CAT scan may be insufficient if the hamartoma atypically lacks cartilage and fat cells. Lung hamartomas are more common in men than in women and may present additional difficulties in smokers.

Some lung hamartomas can compress surrounding lung tissue to a degree, but this is generally not debilitative or even noticed by the patient, especially for the more common peripheral growths. They are conventionally treated by surgical resection.

Tissues affected by lung cancer, tumors, or other malignant airway diseases may be treated with cryofrost to kill tumor cells and effect the replacement of diseased tissue with healthy tissue. Bronchoscopic or laparoscopic techniques may be utilized, depending on the location or size of the tumor.

Lung cancer may also benefit from a cryotherapy enhanced immune response. While not wishing to be bound by any particular theory, it is contemplated that immune effecter cells recruited to the dead and dying cells may develop antibodies that recognize the frozen cancer cells, that cytotoxic T cells may be activated by presentation of tumor cell antigen-derived peptides in association with MHC class I, or that activation of natural killer cells or macrophages may more effectively produce an altered-self attack, such as by recognition of altered MHC expression, following cryotherapy. Not only may such recognition help destroy any surviving cancer cells at the treatment site, such as those in the periphery of the cryofrost, a systemic immune system may also be able to recognize and destroy metastases occurring away from the treated site.

The exact nature and mechanism of the immune response following cryotherapy is deserving of further elucidation. At present, however, there appears to be a ‘freezing stimulated’ change in immune response such that cryotherapy can be thought of as a primer for up regulating the immune system when used in conjunction with immunotherapy—such as administration of macrophages or dendritic cells, which may be harvested from the patient's own bone marrow or blood and cultured with appropriate growth factors (possibly to facilitate maturation), or systemic chemotherapy. As such, combination therapy with cryotherapy and one of the above or other modalities appears to generate antimetastatic effects and functional antitumor memory. Therefore, the ability to generate systemic immune responses in the pleural space, airways or elsewhere could be a may be an effective treatment, characterized by cryospray administered at the same time or followed by administration of antigen presenting cells or the delivery of systemic chemotherapy.

Additionally, as some cancer cells may survive cryoablation and cause cancer recurrence, it is further contemplated that anti-cancer gene therapy may be used in combination with the cryosurgical procedure. For example, tumor suppressor genes or genes that promote apoptosis of the cancer cells may be administered.

Lung Infections

Lung infections may be caused by any pathogenic organism, such as bacteria, fungi, viruses, or parasites. While not wishing to be bound by any particular theory, it is contemplated that cryotherapy may be used to kill pathogens by freezing them and/or activating a cold shock response that inhibits growth and pathogenesis. It is further contemplated that cryotherapy may also be used to stimulate an innate or homoral immune response, thereby signaling immune effecter cells to respond and fight the source of infection. Infections in the chest lead to inflammation which often times results in a unregulated wound response resulting in progressive injury not only to the lungs but to the rest of the body—i.e. a sepsis syndrome. Cryogen can be used to dampen the inflammatory response as well as for direct insult to the offending pathogen thus restoring the appropriate host response and establishing control of the infectious agent.

Pleurisy

The lungs and chest cavity are lined with thin membranes called pleura. With each breath, the pleura slide smoothly against each other, lubricated by a fluid. Pleurisy occurs when the pleura become inflamed and they rub and grate against each other. This causes pain, aggravated by coughing and deep breathing. Also called pleuritis, the inflammation is often caused by respiratory illnesses, including tuberculosis, pneumonia, and asbestos-related diseases. Other causes include viral and bacterial infections and rheumatic conditions like lupus erythematosus. Symptoms include a recent or existing respiratory infection, persistent cough, chest pain, pain when breathing deeply or coughing, malaise, and fever.

Sometimes the inflammation can lead to a collection of fluid between the pleura, called pleural effusions. A collection of pus in the pleural cavity is called empynema. The fluid buildup is either caused by one membrane creating excess fluid or one membrane failing to drain the fluid. Pleural effusions ease the pain by cushioning between the inflamed membranes, leading the patient to believe that the condition is improving when it actually may be getting worse. A large accumulation of fluid can compress the lungs and cause breathing difficulties, coughing, and cyanosis.

It is contemplated that cryotherapy can be applied to both reduce inflammation and kill any instigating pathogens. The pleural space may be drained and laparoscopic techniques may be used to administer cryospray to the pleural tissues sufficient to kill the infectious agent but not substantially damage the tissue and may also counteract the heat associated with inflammation.

Tuberculosis (TB)

Tuberculosis (TB) is an airborne infection caused by the bacterium Mycobacterium tuberculosis that primarily affects the lungs. TB can be spread by coughing, sneezing, laughing or singing. Repeated exposure to someone with TB disease is generally necessary for infection to take place. Although TB primarily affects the lungs, other organs and tissues may be affected as well.

Multidrug-resistant tuberculosis (MDR TB) is a form of tuberculosis that is resistant to two or more of the primary drugs (isoniazied and rifampin) used for the treatment of tuberculosis. Extensively drug-resistant TB (XDR TB) is TB resistant to at least isoniazied and rifampin among the first-line anti-TB drugs, and among second-line drugs, is resistant to any fluoroquinolone and at least one of three injectable drugs. Resistance to one or several forms of treatment occurs when the bacteria develop the ability to withstand antibiotic attack and pass on that ability to newly produced bacteria. Since that entire strain of bacteria inherits this capacity to resist the effects of the various treatments, resistance can spread from one person to another. On an individual basis, however, inadequate treatment or improper use of the anti-tuberculosis medications remains an important cause of drug-resistant tuberculosis. Drug-resistant TB is difficult and costly to treat and can be fatal.

Cryotherapy may be helpful in killing the bacteria throughout the lung that cause TB. Additionally, damaged or diseased tissue or entire lobes may be removed with ablation by cryosurgery.

Pneumonia

Pneumonia is characterized by inflammation and flooding of the alveoli with fluid. Pneumonia can result from a variety of causes, including infection with bacteria, viruses, fungi, or parasites, and may also result from chemical or physical injury to the lungs. Pneumonia is also commonly a symptom developed as a result of another type of lung disease.

There are several different types of pneumonia that originate from different causes. For example, severe acute respiratory syndrome (SARS) is a highly contagious and deadly type of pneumonia which first occurred in 2002 after initial outbreaks in China. SARS is caused by the SARS coronavirus, a previously unknown pathogen. Bronchiolitis obliterans organizing pneumonia (BOOP) is caused by inflammation of the small airways of the lungs. It is also known as cryptogenic organizing pneumonitis (COP).

Eosinophilic pneumonia is invasion of the lung by eosinophils, a particular kind of white blood cell. Eosinophilic pneumonia often occurs in response to infection with a parasite or after exposure to certain types of environmental factors.

Chemical pneumonia (usually called chemical pneumonitis) is caused by chemical toxins such as pesticides, which may enter the body by inhalation or by skin contact. When the toxic substance is an oil, the pneumonia may be called lipoid pneumonia.

Aspiration pneumonia is caused by aspirating foreign objects, usually oral or gastric contents, either while eating, or after reflux or vomiting which results in bronchopneumonia. The resulting lung inflammation is not an infection but can contribute to one, since the material aspirated may contain anaerobic bacteria or other unusual causes of pneumonia. Aspiration is a leading cause of death among hospital and nursing home patients, since they often cannot adequately protect their airways and may have otherwise impaired defenses.

Pneumonia is commonly treated with oral antibiotics. However, cases caused by resistant strains of bacteria may require hospitalization and IV administration of newer antibiotics. It is contemplated that cryotherapy may be beneficial in treating minor and severe cases of pneumonia by freezing or killing pathogens. Cryotherapy may be especially useful in treating patients infected with pathogens that are drug resistant or patients who can not tolerate antibiotic drugs. Cryotherapy may also stimulate an enhanced immune response that can help destroy pathogens.

Occupational Lung Disease

Occupational lung disease is the number one work-related illness in the United States based on the frequency, severity, and preventability of diseases. In severe cases, it can develop into ILD. These illnesses are usually caused by extended exposure to irritating or toxic substances that may cause acute or chronic respiratory ailments, although severe single exposures can cause chronic lung disease as well. It is characterized by permanent alteration of lung structure caused by inhalation of a mineral dust and the reaction of the lung tissue to this dust. The reactions that occur within the lungs vary with the size of the dust particle and its biological activity. While some dusts (like barium, tin, and iron) do not result in a fibrogenic reaction in the lungs, others can evoke a variety of tissue responses. Such responses include nodular fibrosis (silicosis), diffuse fibrosis (asbestosis), and macule formation with focal emphysema (coal worker's disease). Still others (like beryllium) can evoke a systemic response and induce a granulomatous reaction in the lungs.

Occupational lung diseases are often associated with Pneumoconiosis, also known as coal workers' pneumoconiosis, dust disease, miner's asthma, or black lung disease. Pneumoconiosis is caused by the inhalation of coal dust, characterized by formation of nodular fibrotic changes in lungs. These changes may be in the form of industrial bronchitis, a condition which abates 3 to 6 months following the cessation of exposure, or permanent changes in the lung parenchyma, taking the form of macules, micronodules, macronodules, or progressive massive fibrosis. Pneumoconioses can appear and progress after the exposure has ceased. Regression does not occur, and treatment is mostly symptomatic and supportive. Smoking can act synergistically to increase the severity of these diseases.

In 2002, there were about 294,500 newly reported cases of occupational illness in the private industry, and 22,000 newly reported respiratory conditions. Overall, 2.5 per 10,000 full time workers developed nonfatal occupational respiratory diseases.

For example, Black lung (coal workers' pneumoconiosis) is a lung disease caused by deposits of coal dust in the lungs. Black lung results from inhaling coal dust over a long time. Although coal dust is relatively inert and does not provoke much reaction, it spreads throughout the lungs and shows up as tiny spots on an x-ray. Coal dust may block the airways. In simple black lung, coal dust collects around the small airways (bronchioles) of the lungs. Every year, 1 to 2% of people with simple black lung develop a more serious form of the disease called progressive massive fibrosis, in which large scars (at least ½ inch in diameter) develop in the lungs as a reaction to the dust. Progressive massive fibrosis may worsen even after exposure to coal dust stops. Lung tissue and the blood vessels in the lungs can be destroyed by the scarring.

Cryotherapy may be used to remove macules, micronodules, macronodules, and fibrous tissue and stimulate regeneration of healthy tissue. In patients in which foreign particles remain within the lung, ablation of contaminated regions may also help stimulate immune system cells, including re-ciliation of epithelials, to clear the particles from treated tissues.

Pulmonary Vascular Diseases

It is contemplated that cryotherapy may also be useful in the treatment of pulmonary vascular diseases such as Primary Pulmonary Hypertension (PPH), Secondary Pulmonary Hypertension (SPH), Pulmonary Vasculitis and Alveoloar Hemorrhage Syndromes, and Pulmonary Embolism.

The cryogen would dampen the proliferative response seen in the pulmonary arterioles as well as provide some thinning to the vessels such that their normal caliber might be restored and that further loss of luminal diameter may be stopped.

Drug Induced Lung Disease

Drug-induced lung disease is a major source of iatrogenic injury. Awareness of drug-induced pulmonary disease is increasing: a review published in 1972 identified only 19 drugs as having the potential to cause pulmonary disease; now at least 150 agents are recognized, and the list continues to grow. Drug-induced pulmonary disease is lung disease typically caused by a bad reaction to a medication. Many types of lung injury can result from medications, for example, allergic reactions (e.g., asthma, hypersensitivity pneumonitis, or eosinophilic pneumonia), alveolar hemorrhage (bleeding into the lung air sacks), bronchitis, drug-induced lupus erythematosus, granulomatous lung disease (i.e., a type of tumor in the lungs, inflammation of the lung air sacks (e.g., pneumonitis or infiltration), interstitial fibrosis, lung failure, lung vasculitis (i.e., inflammation of lung blood vessels), mediastinitis, pulmonary edema, pleural effusion, and swollen lymph nodes. Numerous drugs are known to cause lung disease in some people, including those used during chemotherapy and to treat certain heart conditions. Other drugs known to cause lung disease in some people include certain antibiotics and illicit drugs.

Drug-induced lung disease may be identified using a variety of tests including, but are not limited to, bronchoscopy, chest CT scan, chest x-ray, lung biopsy, and thoracentesis.

Cryogen can be used to dampen the inflammatory response as well as for ablation of damaged tissue and stimulation of tissue regeneration.

Radiation Pneumonitis

One embodiment of the invention relates to treating and/or preventing Radiation pneumonitis. Radiation pneumonitis is a type of inflammatory response of the lung tissue in response to radiation insult, and is characterized by lymphocytic alveolitis, a result of inflammatory infiltrates of mononuclear cells from the vascular compartment into the alveolar spaces. As expected at sites of inflammation, an active interaction between cellular and humoral factors are involved including immune cells, parenchymal cells, macrophages, chemokines, adhesion molecules, lymphocytes, inflammatory cytokines and fibrotic cytokines. Radiation-induced pneumonitis is a familiar complication of therapeutic radiation exposure of tumors, and the adverse side effects associated with such therapeutic regimens interfere with the ability of patients to continue on a therapeutic regimen and oftentimes result in dose reduction or dose interruption.

The present invention provides methods useful for treating and/or preventing radiation pneumonitis comprising contacting the tissue with a cryogen, or using the cryogen to create an isotherm in proximity to the tissue.

In one embodiment of the invention, methods for treating and/or preventing Radiation pneumonitis comprising contacting the tissue with a non-cryogenic gas, or using the non-cryogenic gas to create an isotherm in proximity to the tissue are provided.

In some embodiments, the cryogen or the non-cryogenic gas is sprayed directly onto the area afflicted with radiation pneumonitis.

Respiratory Distress Syndrome (ARDS)/Bronchopulmonary Dysplasia (BPD)

ARDS is a severe lung disease caused by a variety of direct and indirect insults. It is characterized by inflammation of the lung parenchyma leading to impaired gas exchange with concomitant systemic release of inflammatory mediators causing inflammation, hypoxemia and frequently resulting in multiple organ failure. This condition is life threatening and often lethal, usually requiring mechanical ventilation and admission to an intensive care unit. A less severe form is called acute lung injury (ALI). ARDS can be caused by any major lung inflammation or injury. Some common causes include pneumonia, septic shock, trauma, aspiration of vomit, or chemical inhalation.

Bronchopulmonary Dysplasia (or BPD) is a chronic lung disease with persistent difficulty breathing and abnormal changes on the chest X-ray, that sometimes follows lung diseases that affect newborn infants. It is characterized by inflammation and scarring in the lungs. In most cases, BPD occurs in infants who are born prematurely and who have Respiratory Distress Syndrome (or RDS), a lung disease common in premature babies. In some cases, BPD may follow other lung conditions of the newborn, such as pneumonia. In most cases, BPD occurs after babies have required extra oxygen and/or a mechanical ventilator to treat their original lung problem. In many cases, the symptoms of BPD disappear quite rapidly. Some infants with BPD may have breathing difficulties for many months or years.

Application of cryogen may be used to dampen the inflammatory response observed in these conditions.

### EXAMPLES

In the performed studies, a 7Fr ERCP-like catheter was utilized and inserted through the biopsy tube of a standard therapeutic bronchoscope. The cryogen that was used was liquid nitrogen.

### Example 1

### CryoSpray Ablation of Swine Airway

A first study was performed to assess the efficacy and safety of this utilizing cryospray ablation in the airway of a swine. Using a straight tip catheter cryospray ablation was initiated multiple times in the primary bronchus. The swine was monitored continuously for respiratory conditions such as barotrauma via fluoroscopy. Bleeding was manually stimulated via a biopsy forceps injury to assess the effect of the cryospray ablation on the injury.

The entire right bronchus was treated in approximately 10 seconds. No barotrauma was seen, but slight hyperemia was noted. Following the procedure, two tissue samples were taken from the airway of the swine. A first sample was taken at thirty-five minutes post-cryospray ablation. A second tissue sample was taken at sixty minutes post cryospray ablation. Significant pathological findings in biopsies taken 35 minutes post treatment include an absence of surface mucosa, tissue consisting mostly of submucosal glands, and intact connective tissue largely resistant to treatment. Additionally, pathology findings in biopsies taken 60 minutes post-treatment include a pronounced injury to the cells with a measurable depth of injury indicating a fatal injury to the tissue

### Example 2

### CryoSpray Ablation of Swine Airway

In Study 2, fifteen specimens (swine) were utilized. Twelve specimens were utilized in the study to eliminate intersubject variability, while three specimens were held as replacement specimens. Each swine was male and the average weight of the test animals was one hundred fifty (150) pounds.

The twelve swine were broken down into four treatment subgroups:

GROUP 1 (3 specimens)—theoretical barotrauma limit, taken to failure (acute).

GROUP 2 (3 specimens)—these specimens were subjected to four cycles of 5 second cryospray ablation on day zero. Each of the specimens was observed and biopsied on days 2, 4, 7. The specimen was recovered on day 7. No biopsy was taken on the day of euthanization. The specimens of this group were euthanized while under general anesthesia.

GROUP 3 (3 specimens)—these specimens were subjected to four cycles of 5 second cryospray ablation on day zero. The first specimen was observed and biopsied on day 2. A second different specimen was observed and biopsied on day 4. A third specimen was observed and biopsied on day 7. The specimen were recovered on day 28. The specimens of this group were euthanized while under general anesthesia. The specimens received only one post-operative observation and biopsy to limit stress levels during the 28 day recovery period.

GROUP 4 (3 specimens)—these specimens were subjected to two cycles of 5 second cryospray ablation on day zero (0). Each specimen was observed and biopsied on days 2, 4, 7. The specimen was recovered on day 7. No biopsy was taken on the day of euthanization. The animals of this group were euthanized while under general anesthesia.

General Procedure

All procedures performed on the test specimens were done under general anesthesia. General anesthesia induction was performed with a Telazol Cocktail given intramuscularly (IM). The specimen was then intubated with an appropriately sized cuffed endotracheal tube (ET). An IV catheter was then placed in the marginal ear vein or other appropriate vein as necessary. Intravenous fluids (Lactated Ringers Solution, LRS) were administered to the specimen at a rate of 10 ml/kg/hour. Approximately 30 to 60 minutes prior to the cryospray ablation procedure each specimen was given a standard dose of glycopyrrolate to reduce secretions.

The surgical procedure took between 60 and 90 minutes per specimen. All of the surgical procedures were performed with a standard therapeutic bronchoscope. The endoscope was fitted with either a straight spray and/or directional tip catheter for the application of the cryospray utilizing the cryospray device described above. Group one received treatment proximal to the main carina using a straight tip catheter to ensure even distribution of pressure between the left and right lung. Groups 2, 3, and 4 received treatment with a directional tip catheter four centimeters distal to the main carina to ensure uniform and consistent treatment of an approximate two centimeter by 90 degree treatments area.

GROUP 1 Procedure and Results—Safety failures and determination failure mode were attempted by the physician. Failure modes included airway disruption, bleeding, barotrauma, severe cardiovascular disruption and death. The catheter was inserted into the pulmonary artery to measure pressure. The ET Cuff was deployed, the vent circuit was closed and the specimen was actively ventilated. The endoscopic procedure was performed using a straight tip catheter. The liquefied gas (i.e., cryogen) was applied proximal to the primary carina. A necropsy was performed where the following organs were removed and examined grossly: liver, spleen, kidney, lung, heart, the cryo treatment site (including at least 2 centimeters in circumference around the visible cryo-injury). Abnormal specimens were stored for future analysis per standard operating procedure and based on the failure. Specimens may also be taken for histological review and fixed in formalin. The failure mode analysis yielded no evidence of airway disruption in any of the animals tested. Cardiac compromise and system failure resulted from the extreme intrathoracic pressure challenge.

GROUPS 2 and 3 Procedure and Results—The specimens were biopsied and observed at multiple time points post-operatively in order to determine the degree of tissue healing at the injury site. The cryospray endoscopy was performed were the ET cuff was deflated and the vent circuit opened during a five (5) second spray period. Four (4) cycles were performed for five (5) seconds for a total treatment time of 20 seconds. The five (5) second interval initiates at the first appearance of cryofrost. The cryospray was directed at the lateral wall of a specimen's lung where the physician maintained a constant focal point throughout the cryospray spray time. A minimum thaw period of 60 seconds was allowed between each cryospray treatment. A biopsy was also taken at the treatment site, at a location 180 degrees from the focal treatment site and from the uninjured bronchus. The lung was assessed for contralateral injury at all biopsy intervals. The biopsies were repeated for Group 2 at days 2, 4. The biopsies were repeated for Group 3 at days 2, 4 and 7.

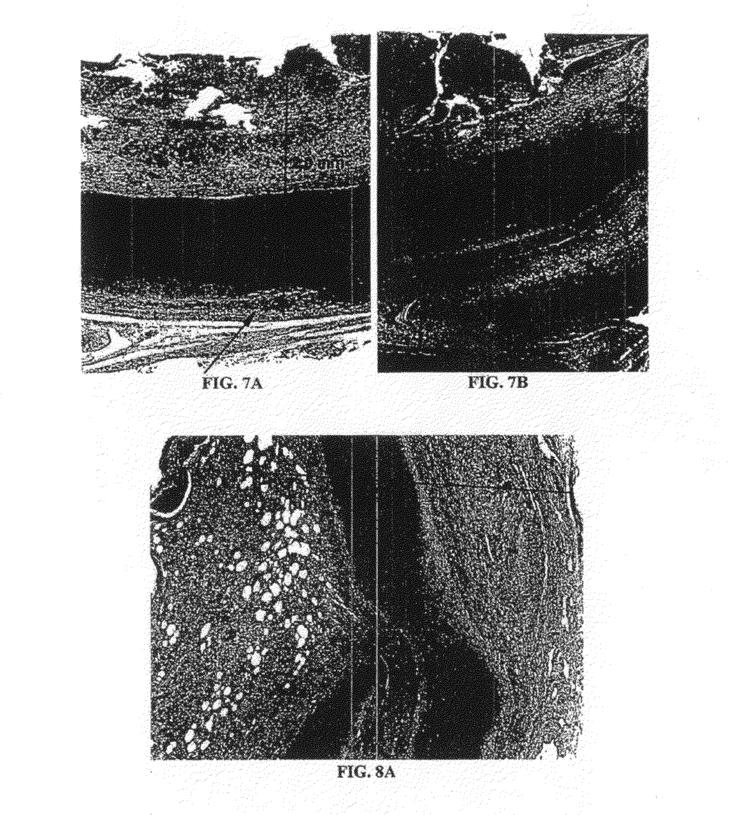

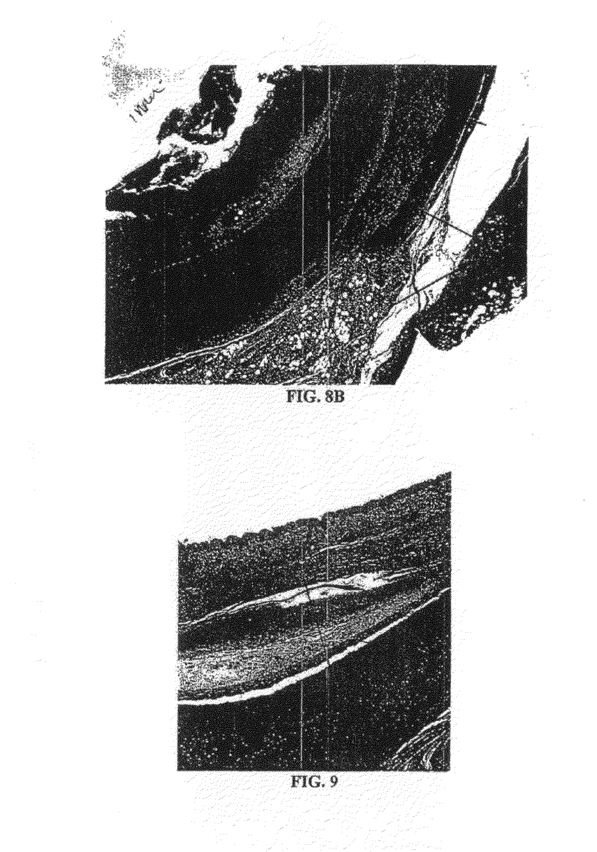

Each of the Group 2 specimens were euthanized immediately subsequent to the evaluation on day 7 and a complete necropsy was performed to harvest the cryo-treatment site (including at least 2 centimeters in circumference around the visible cryo-injury). The cryo-injury was photographed and videotaped to determine the extent of the ablation and the presence of complications. The specimen was prepared according to pathology. The three treatment animals yielded relatively consistent injury at the site of directed cryo-spray. There were no obvious adverse effects; the animals tolerated the procedure well and without complications. Treated sites exhibited slight erythema, tissue sloughing of the mucosa as well as healing within 1 week of treatment. Visual and histologic examination on days 2 and 4 post treatment revealed gross mucosal injury and evidence of emergent reepithelialization. There was no visual evidence of scarring in any of the airways examined. Histologic inspection revealed the treatment effect was confined to the treatment site and to a lesser extent, the contra-lateral region of the treated bronchus. FIGS. 7A and 7B show a side-by-side comparison of the extent of damage between 4 cycles of 5 seconds and 2 cycles of 5 seconds. FIGS. 8A and 8B. show the histological findings of maximum depth injury. The specimen shown in FIG. 8A presents with pus, ulceration, acute inflammation, and obliteration of smooth muscle and glands. The depth of injury is to adventitia, about 2.8 mm and about 15 mm long. FIG. 8B shows extensive and deep injury with ulceration and neutrophiles at an approximate depth of 3 mm.

Each of the Group 3 specimens were euthanized immediately after the evaluation on day 28. The cryo-injury was photographed and videotaped to determine the extent of the ablation and the presence of complications. A necropsy was performed harvesting the cryospray treatment site (including at least 2 cm in circumference around the visible cryo-injury. The specimen was prepared according to pathology. Histological evaluations of representative sections were determined to determine the depth of the ablation. Complete reepithelialization and normalization of the tissue was seen at day 28, with the exception of the cartilaginous layers up to a depth of about 2.7 mm, which may need additional time to heal due to lack of vascularity (FIG. 9). FIG. 10 also shows extent of healing at day 28, with prior cartilage plate damage evident at a depth of 2.4 mm. Chondrogenesis can also be seen at the periphery of the cartilage.

GROUP 4 Procedure—The specimens of this group was biopsied and observed at multiple time points post-operatively in order to determine the tissue healing at the injury site. The cryospray endoscopy was performed where the ET cuff was deflated and the vent circuit opened during the five second spray period. Two cycles were performed for five seconds for a total treatment time of ten seconds. The five second interval initiates at the first appearance of cryofrost. The cryospray was directed at the lateral wall of a specimen's lung where the physician maintained a constant focal point throughout the cryospray spray time. A minimum thaw period of sixty seconds was allowed between each cryospray treatment. A biopsy was also taken at the treatment site, at a location 180 degrees from the focal treatment site and from the uninjured bronchus. The lung was assessed for contralateral injury at all biopsy intervals. The biopsies were repeated for Group 2 at days 2, 4.

Each of the Group 4 specimens were euthanized immediately subsequent to evaluation on day 7 and a complete necropsy was performed to harvest the cryo-treatment site (including at least 2 centimeters in circumference around the visible cryo-injury). The cryo-injury was photographed and videotaped to determine the extent of the ablation and the presence of complications. A histological evaluation was also performed of the representative sections of the cryo-treatment site to determine the depth of the ablation. The three treatment animals yielded relatively consistent injury at the site of directed cryo-spray. There were no obvious adverse effects; the animals tolerated the procedure well and without complications. Treated sites exhibited slight erythema, tissue sloughing of the mucosa as well as healing within 1 week of treatment. Visual and histologic examination on days 2 and 4 post treatment revealed gross mucosal injury and evidence of emergent reepithelialization. There was no visual evidence of scarring in any of the airways examined. Histologic inspection revealed the treatment effect was confined to the treatment site and to a lesser extent, the contra-lateral region of the treated bronchus.

Pathology—The specimen's right stem was resected to evaluate the treatment area and the left stem was resected as a control. The medial wall of the right stem was marked. A cut was made along the medial wall to allow observation of the inside of the lateral wall. The approximate center of the injury was identified and two vertical orientation markers were placed at least 2 cm from the injury site along the horizontal centerline of the injury. An X-Y axis horizontal template was placed over the specimen, such that the approximate center of the focal injury was positioned with the center axis of the X-Y grid. The orientation markers were used to align the grid and to allow the reviewing pathologist the same approximate positioning for the individual histological sections, therefore, the markers remained in the specimen. The specimens were preserved in formalin and the X-Y grid template and specimen were sent to the pathologist for review. Upon receipt of the specimen, the pathologist performed gross observations of each specimen and prepared slides based on the X-Y grid template. A slide was prepared for the center of the injury, and for each 0.5 cm distance from the center line in both the vertical and horizontal directions. The horizontal axis is important so the entire length of the horizontal axis was analyzed in 0.5 cm increments. The observed tissue farthest from the center line of each slide along the vertical axis showed little or no effect from the treatment. As a result, the top and bottom 3 cm portions from the centerline were evaluated. Slides were prepared and each of the specimen evaluated to assess the degree of the injury biopsied. Adequate ablation was qualified by evaluating the tissue reaction at the treatment site for inflammation, hemorrhage, cryonecrosis and depth of the injury.

Freezing of the respiratory tissue was recognizable by a white “cryo-burn” with sharply demarcated margins. This was followed by slow thawing within minutes and then a sloughing off of the ablated tissue in the subsequent 7 day period. (FIG. 11)

These experiments on living swine, which are a valid model of the human respiratory tract and airways, demonstrate the safety and efficacy of cryotherapy in swine. Preliminarily, they suggest feasibility for thoracic applications in the treatment of benign and malignant human lung disease.

### Example 3

Cryospray Ablation™ Using Surgical Resection Specimens to Determine Safety and Histological Effect in the Lung (CSAir 1).

Methods: CSA was administered in healthy airway tissue of 21 subjects during a standard bronchoscopy prior to lung resection. Group 1 (n=5) received same day therapy, Group 2 (n=2) 2-4 days, Group 3 (n=3) 5-7 days, and Group 4 (n=5) 8+ days, prior to lobectomy. All groups received 2 cycles of 5 second spray dosimetry with a 60 second interim thaw. Oxygen saturation and peak airway pressure were monitored constantly. Subjects received 100% oxygen through out the procedure. Histological inspection of the resected specimen was preformed by a blinded pathologist.

Results: No adverse events were reported. 5 of the 21 subjects did not undergo resection. Histologic examination of Groups 1 and 2 revealed loss of epithelium and muscularis mucosa, edema, and damaged sub-mucosal glands. Group 3 revealed areas of denuded mucosa at the treatment site, but showed adjacent re-epithelialization, although edema and loss of smooth muscle and glands were still evident. One specimen in Group 4 showed complete re-epithelialization and normalization of tissue except some residual edema and some permanent loss of smooth muscle. Depth of cryo-necrosis in all groups was limited to the mucosal and submucosal layers (˜0.5 mm), with no evidence of connective tissue injury. There was no evidence of scarring in any of the airways examined.

Conclusions: The results of this trial demonstrate the safety and efficacy of CSA in the human airway.

Materials and Methods

The protocol for this clinical study was approved by the MedStar Health System IRB. Informed consent was obtained from all subjects prior to their participation in the study.

Application of CSA Therapy

The study consisted of 4 groups of subjects with group being sprayed at a specific time interval prior to operative intervention with curative intent for reasons unrelated to the study.

Group 1 (n=5) received treatment on the same day as their operation, Group 2 (n=2) 2-4 days prior to operative intervention, Group 3 (n=3) 5-7 days prior to operative intervention, and Group 4 (n=5) 8+ days prior to operative intervention to assess tissue destruction and healing at various time points. There was some variability in the numbers of patients enrolled in each group in order to accommodate procedure schedules. Subjects were scheduled for FFB with cryospray application during routine pre-operative bronchoscopy between 1 hour and 106 days prior to scheduled surgery, as determined by the group assignment. Institutional guidelines for bronchoscopy regarding sedation and analgesia were followed for the procedures. Staff investigators performed the FFB using a video bronchoscope (Olympus BF-X1T160 or BF-X1T180) with adherence to standard protocol. Once a target site for the application of the cryogen was selected, a dye marker was placed proximal to the intended treatment area so the spray location could later be identified in the resected specimen. All groups received a treatment dosimetry of 2 cycles of 5 second spray dosimetry with a 60 second thaw interval. Application of the cryospray was targeted to visually accessible airways distal to the proposed area of resection which essentially meant delivery of the cryogen to distal lobar and segmental bronchi.

It is important to note that this modality is a non-contact method of ablation; the cryocatheter does not touch the airway wall. Subjects were placed on 100% oxygen during the procedure and oxygen saturation and peak airway pressure were monitored throughout the procedure. Subjects were treated with narcotic analgesics and anti-emetics as needed after each procedure. Additionally, subjects were interviewed using a standardized questionnaire prior to the procedure to specifically solicit symptoms present before cryospray therapy as a baseline to which to compare symptoms exhibited after application of the cryogen. Subjects in Groups 24 were contacted by telephone one day following cryo application, as well as 2-7 days post treatment to complete the standardized quesstionairre and assess any side effects or complications.

Resection specimens were examined histologically by a pulmonary pathologist blinded to all clinical information except the anatomic location of the specimen.

Lung specimens were fixed by distension with 10% formalin solution to facilitate dissection. Two- to 3-millimeter-thick sections of treated airways were obtained serially along the length of the excised segment, and the orientation of tissue slices was preserved. Samples were also obtained proximal and distal to the treated areas, and slides were prepared with hematoxylin-eosin stain. Observations of the airway wall and surrounding tissue were noted, with special attention to the mucosa, sub-mucosa, muscularis propria, and cartilage.

Results

Subjects were enrolled and treated between December 2007 and May 2008. The treated patients included 11 male and 10 female patients ranging in age from 38 to 82 years (mean age, 60±11.4 years SD). All subjects received cryospray therapy and went to surgery for resection. As a consequence of intraoperative findings only fifteen subjects had complete anatomic resections. Group assignments are shown in Table 1.

TABLE 1

| | | | | Time between | | |
|---|---|---|---|---|---|---|
| Subject | | | Group | CSA treatment | | Resection |
| Number | Gender | Age | Assignment | and resection | | completed? |
| 01-001 | M | 53 | Group 3 | 7 | day | Yes |
| 01-002 | F | 82 | Group 3 | 7 | day | Yes |
| 01-003 | M | 57 | Group 4 | 14 | day | No |
| 01-004 | F | 67 | Group 4 | 106 | day | Yes |
| 01-005 | F | 68 | Group 3 | 5 | day | No |
| 01-006 | M | 60 | Group 3 | 5 | day | Yes |
| 01-007 | M | 71 | Group 1 | >1 | day | Yes |
| 01-008 | M | 61 | Group 1 | >1 | day | Yes |
| 01-009 | M | 72 | Group 3 | 7 | day | No |
| 01-010 | F | 38 | Group 2 | 2 | day | Yes |
| 01-011 | F | 43 | Group 1 | >1 | day | Yes |
| 01-012 | F | 55 | Group 2 | 2 | day | Yes |
| 01-013 | M | 56 | Group 2 | 3 | days | No |
| 01-014 | M | 51 | Group 4 | 30 | days | No |
| 01-015 | F | 77 | Group 4 | 11 | day | Yes |
| 01-016 | M | 56 | Group 1 | >1 | day | Yes |
| 01-017 | F | 77 | Group 4 | 12 | day | Yes |
| 01-018 | M | 56 | Group 1 | >1 | day | No |
| 01-019 | F | 50 | Group 1 | >1 | day | Yes |
| 01-020 | F | 68 | Group 4 | 22 | day | Yes |
| 01-021 | M | 69 | Group 4 | 19 | day | Yes |

There were no adverse events reported with any of the procedures. No side effects were noted which were not primarily attributable to the bronchoscopy itself, including sore throat, or hoarseness. Subjects reported no pain after treatment, as was evidenced by the absence of requests for additional medications (i.e., antibiotics, bronchodilators, anti-inflammatory medications) or supplemental oxygen. There were no additional or unscheduled visits of any of the study subjects to health-care providers as a result of treatment. Subjects who did not undergo resection were contacted by study personnel, and given a number to call should any signs or symptoms arise that could be related to CSA treatment. No subjects called to report symptoms or side effects.

The time between CSA therapy and surgical resection ranged from 1 hour to 106 days. All CSA treatments were completed in less than 5 minutes at the conclusion of the preoperative bronchoscopy. The application regimen was a total of 10 seconds (2 cycles of 5 second sprays) in all subjects with a minute of thaw time after each application of cryogen. Cryospray was directed at one area in the airways subtending the proposed area of resection. Subjects in Groups 2-4 were contacted by telephone one day following CSA treatment, and 2-7 days post treatment to assess any side effects or complications. They were asked the same question set solicited at baseline to determine changes in normal pain or symptom patterns.

There were no adverse events reported with any of the procedures and no side effects were noted which were not primarily attributable to the bronchoscopy itself, including sore throat, or hoarseness. Subjects reported no pain after treatment, as was evidenced by the absence of requests for additional medications (ie, antibiotics, bronchodilators, anti-inflammatory medications) or supplemental oxygen. There were no additional or unscheduled visits of any of the study subjects to health-care providers as a result of treatment.

Histology

Sections of treated airways were examined at 250-μm intervals. Sections for histologic review included areas of untreated tissue since samples were taken proximal and distal to the treated areas. Findings from the treated areas were consistent with the type of tissue response (both injury and healing) seen in our previous observations on the swine models. These findings were anticipated given our previous observations on the effects of CSA therapy on airways of swine as shown in the preceding examples.

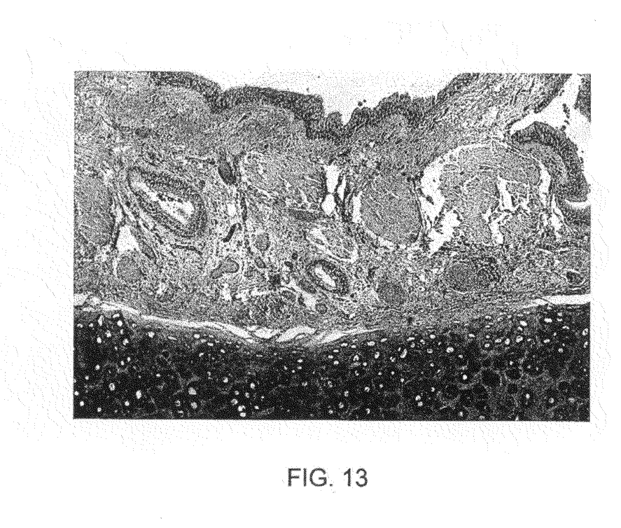

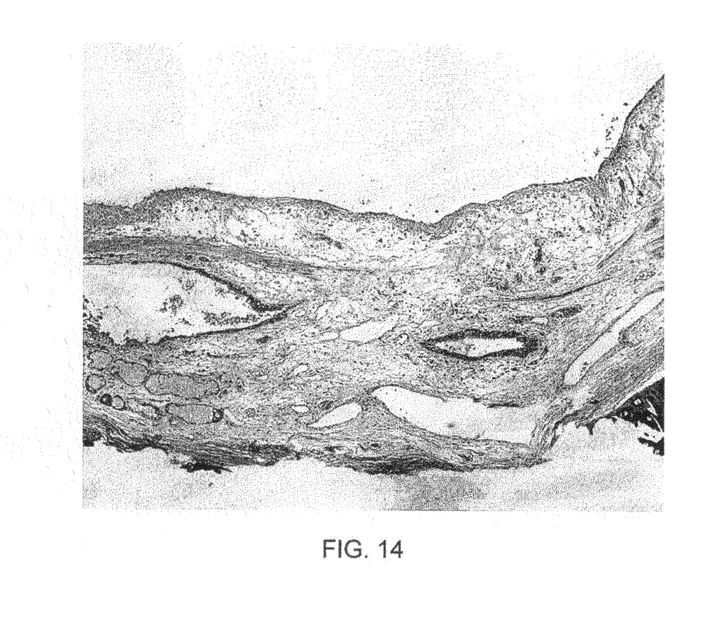

Histologic changes from baseline (FIG. 13) in Groups 1 and 2 (same day therapy, and 2-4 day therapy, respectively) were robust. Examination revealed loss of epithelium and muscularis mucosa, edema, and damaged sub-mucosal glands. Interestingly, the connective tissue components of the airway epithelium appeared relatively uninjured as opposed to the cellular component of the epithelium which was destroyed (FIG. 14). Further, a dearth of inflammation was noted not only in this group, but also in all groups examined at each time internal.

Specimen resected at Day 5 (Group 3) revealed areas of denuded mucosa at the treatment site, but showed adjacent re-epithelialization and healing from the margin of the injury centrally. The healing of injured tissue was almost complete at Day 7, however, persistent loss of smooth muscle and mucosal glands were still evident after Day 7.

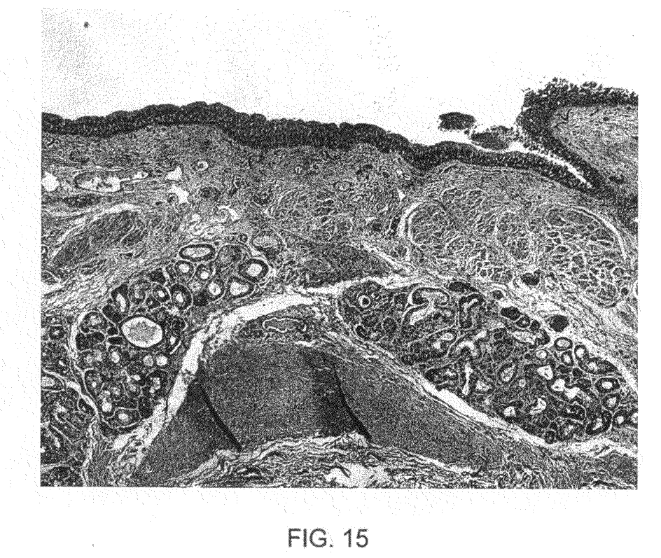

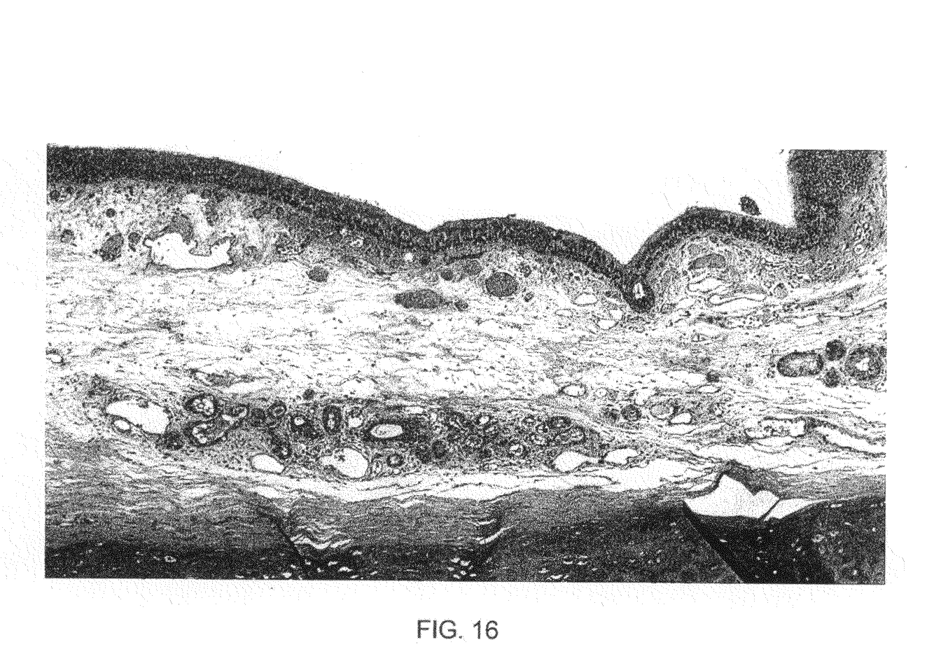

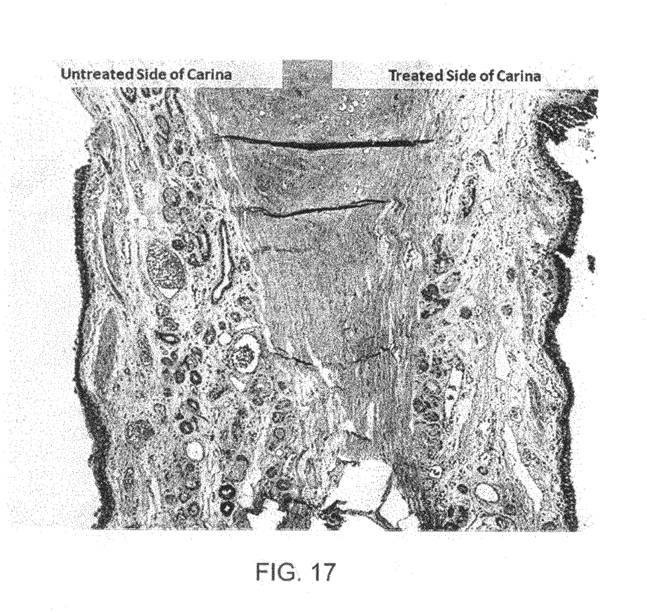

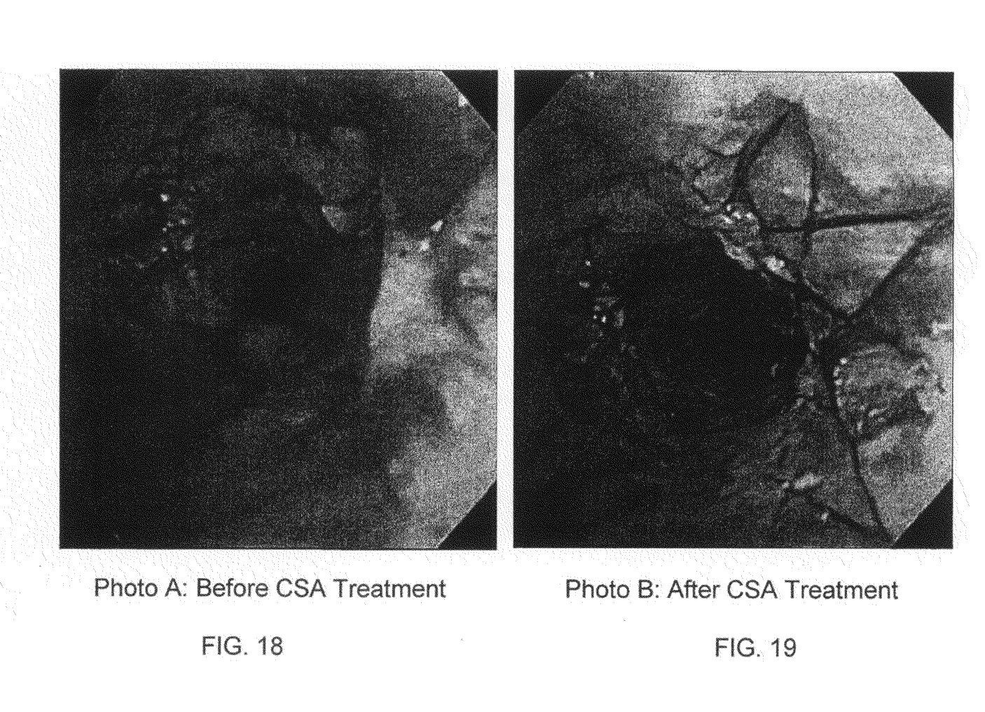

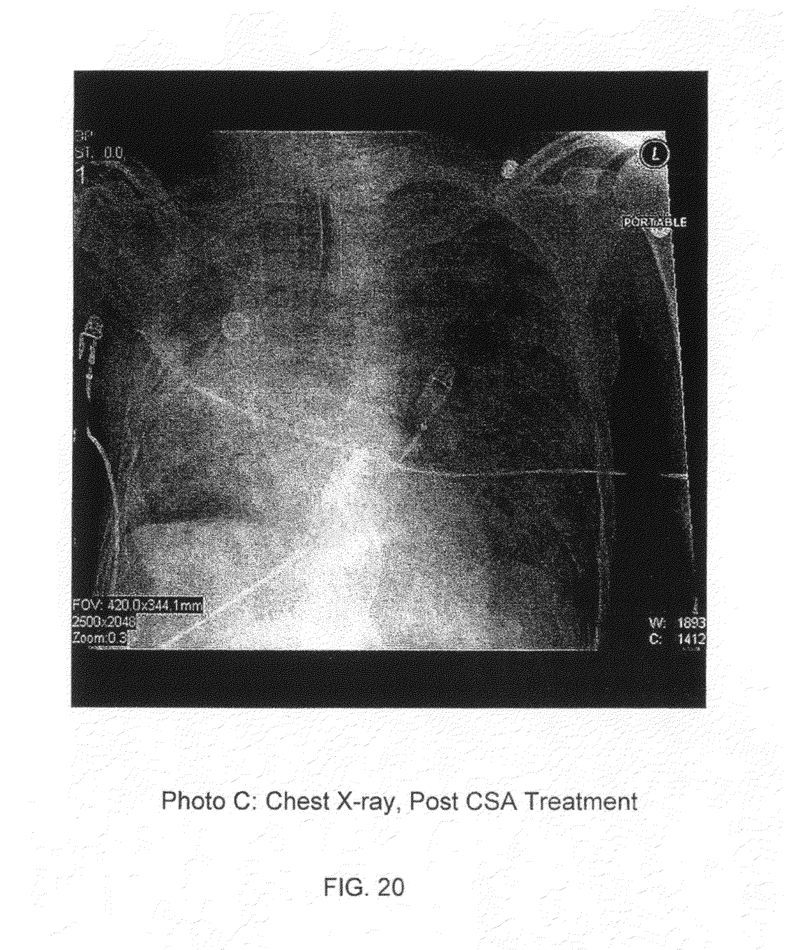

Group 4 (±8 day therapy) demonstrated complete re-epithelialization of the airway mucosa and a thinned or absent smooth muscle layer. A reduction of the glandular layer, or in some cases just thinning of this region, was also noted in group 4. Otherwise normalized mucosa was evident in all 5 Group 4 specimens (FIGS. 15 and 16). The defining feature of this histology was a noticeably thinned muscularis mucosa and the relative absence of glandular tissue when compared with an untreated area in a contralateral airway lumen (FIG. 17). Depth of cryo-necrosis in all groups was limited to the mucosal and submucosal layers (˜0.5 mm), with no evidence of connective tissue injury, including the cartilage. There was no evidence of scarring in any of the airways examined. As mentioned above, the pathologist noted stripping of cellular elements with no evidence of disruption to the connective tissue.

### Example 4

### Stent Overgrowth Treated with Cryospray Ablation

Nearly half of all patients with lung cancer develop some type of airway obstruction. Stenting may initially improve luminal patency, but tissue overgrowth often results, making airway management difficult. Traditional interventions, such as laser and electro-cautery, are risky and potentially fatal. Cryospray ablation (CSA), a non-contact method of destroying unwanted tissue using low-pressure liquid nitrogen, evokes acute and chronic hemostatic effects, leading to regeneration of healthy tissue. This case represents the first use of CSA in the successful treatment of a patient with complete tissue overgrowth of a stent placed to reduce luminal obstruction from lung cancer.

More people die of lung cancer than any other type of cancer. Non-small-cell lung cancers (NSCLC) are the most common and account for about 80% of the total. For early stage lung cancer, surgery remains the mainstay of treatment, and yet a relatively small percentage of these tumors are operable at the time of diagnosis, due either to the advanced stage of the disease or because patient factors prevent resection. The other mainstays of treatment, chemotherapy and radiation, while offering the substantive possibility of remission, are typically not curative and the durability of the results is variable.

Roughly 50% of patients with lung cancer will present with airway involvement at some point during the course of their disease process, with airway obstruction as the catastrophic consequence of this problem. Traditional therapy such as airway stenting, may initially improve luminal patency. However, over time, tissue overgrowth around or through the stent, in the case of either uncovered or partially covered stents, often results, making airway management difficult in the long term. Further, other airway interventions—specifically the thermal modalities, such as laser and electro-cautery—are associated with well-described risks and complications, including death.

Cryospray ablation (CSA) is a non-contact method of destroying unwanted tissue using low-pressure liquid nitrogen. The rapid freezing and thawing of CSA evokes acute and chronic hemostatic effects, as well as acute and subacute forms of intracellular damage, leading to regeneration of healthy tissue. Prior studies of CSA in the airway of swine and humans suggested safety and feasibility for thoracic applications in humans as shown in the preceding examples. This is the first use of the CSA system in the treatment of a patient with complete tissue overgrowth and luminal obstruction of a stent placed initially to reduce luminal obstruction from NSCLC.

Methods: A 54-year-old man with advanced NSCLC was admitted to the intensive care unit with progressive respiratory failure. At the time of admission, a chest X-ray demonstrated complete opacification of the right hemithorax, necessitating intubation and mechanical ventilation. Additionally, the patient required a substantial elevation in the level of supplemental oxygen (60%) to maintain adequate oxygenation. A CT of the chest demonstrated a severe compromise of the airway lumen, starting at the level of the proximal right main stem, with progressive tapering to complete obstruction at the distal end of the bronchus intermedius. Both chest x-rays, and the CT scan also demonstrated the presence of a 40 mm×10 mm stent embedded within the airway mucosa, just below the takeoff of the right upper lobe, extending down into the basilar segments of the right lower lobe.

The patient was brought to the operating room, placed on 100% oxygen, and a bronchoscope was inserted through his endotracheal tube. Once the scope was appropriately positioned in the treatment area, the CSA catheter was deployed through the bronchoscope and CSA therapy was administered. Two cycles of 5-second spray dosimetry with a 60-second interim thaw were administered to each the distal and proximal portions of the treatment area. Total treatment time was approximately 7 minutes. Oxygen saturation and peak airway pressure were monitored throughout the procedure. After suctioning to clear the airways of blood and debris, at least partial luminal patency had been restored and the patient was transported back to the ICU without incident.

Results: There were no adverse events. Prior to the procedure, the patient required 60% FiO₂ on an assist control mode of ventilatory support and had a respiratory rate of between 28 and 32 breaths/minute, along with tidal volumes of roughly 280 ccs. Within 20 minutes of the treatment, ventilatory parameters reflected the change in luminal patency with a respiratory rate of 24-26 breaths/minute, an increase in tidal volume to 350 ccs with the same driving pressure. Supplemental oxygen concentration was able to be reduced to 50% as well.

The patient remained intubated overnight, and a bronchoscopic examination of the area was conducted roughly 18 hours after the treatment. Again, endoluminal debris was cleared from the distal bronchus intermedius. However, sloughing was modest, and further improvement in luminal patency, compared with examination after the initial procedure was noted.

Conclusions: The results of this procedure demonstrate the safety and efficacy of CSA in the human airway, particularly in the presence of stent overgrowth. They also show the speed of the procedure to produce an immediate effect with the attendant decrease in ventilatory support.

The above described examples demonstrate that CSA is effective for a range of human lung diseases.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means and materials for carrying out various disclosed functions may take a variety of alternative forms without departing from the invention. Thus the expressions “means to . . . ” and “means for . . . ” as may be found in the specification above and/or in the claims below, followed by a functional statement, are intended to define and cover whatever structural, physical, chemical or electrical element or structure may now or in the future exist for carrying out the recited function, whether or not precisely equivalent to the embodiment or embodiments disclosed in the specification above; and it is intended that such expressions be given their broadest interpretation.

Modifications may be made without departing from the basic spirit of the present invention. Accordingly, it will be appreciated by those skilled in the art that within the scope of the appended claims, the invention may be practiced other than has been specifically described herein.

A variety of modifications to the embodiments described will be apparent to those skilled in the art from the disclosure provided herein. Thus, the invention may be embodied in other specific forms without departing from the spirit or essential attributes thereof and, accordingly, reference should be made to the appended claims, rather than to the foregoing specification, as indicating the scope of the invention.

What is claimed is:

1. A system adapted to deliver a material to a target tissue in the thoracic cavity, comprising:
a delivery apparatus configured to spray said material onto said target tissue.

2. The system of claim 1, wherein said material is selected from the group consisting of a cryogen, a non-cryogenic gas, a therapeutic agent, a diagnostic agent and a combination thereof.

3. The system of claim 1, wherein said target tissue includes any tissue from the proximal to distal airways and parenchyma.

4. The system of claim 1, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

5. The system of claim 1 adapted for removal of unwanted tissue, removal of an internal or external portion of a lung, or removal of a benign or malignant tumor.

6. The system of claim 1 adapted for modulating an immune response, stimulating chondrogenesis, or treating and/or preventing radiation pneumonitis.

7. The system of claim 1, comprising:
a cryogen delivery apparatus configured to deliver a cryogen to the target tissue, and
an indirect visualization apparatus configured to provide visualization of the target tissue during the cryogen delivery.

8. The system of claim 7, wherein the visualization apparatus and the cryogen delivery apparatus are constructed and arranged to be operationally unintegrated and physically spaced with respect to each other during the delivery of the cryogen.

9. The system of claim 1, comprising:
a cryogen source configured to provide a cryogen,
a regulation apparatus fluidically coupled to the cryogen source and to a cryogen delivery catheter, and
a controller communicatively coupled to the regulation apparatus configured to control the release of cryogen into the cryogen delivery apparatus.

10. A method for treating a tissue in a subject in need thereof, comprising:
identifying the tissue of the subject; and
contacting the tissue with a cryogen, or using the cryogen to create an isotherm in proximity to the tissue.

11. A method according to claim 10, wherein the cryogen is a liquefied gas.

12. A method according to claim 11, wherein the liquefied gas is selected from the group consisting of nitrogen, nitrogen oxides, oxygen, carbon dioxide, liquid air and argon.

13. The method of claim 10, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

14. The method of claim 10, wherein said treating comprises removal of unwanted tissue, removal of an internal or external portion of a lung, or removal of a benign or malignant tumor.

15. The method of claim 10, wherein said treating comprises modulating an immune response, stimulating chondrogenesis, or treating and/or preventing radiation pneumonitis.

16. A method according to claim 10, wherein the tissue is contacted with cryogen or is in proximity to the isotherm for a period of time sufficient to freeze the tissue.

17. A method according to claim 10, wherein a proximal end of a catheter is connected to a cryogen source and a distal end of the catheter is guided to the tissue using a guiding device and cryogen flows from the source through the distal end to the tissue.

18. A method according to claim 17, wherein the guiding device comprises a video camera and the distal end of the guiding device and the catheter is guided to the tissue by observing the distal end of the catheter or guiding device on a video monitor.

19. A method of treating a tissue in a subject in need thereof, comprising:
identifying the tissue of the subject; and
contacting the tissue with a cryogen, or using the cryogen to create an isotherm in proximity to the tissue, and
administering a therapeutic agent.

20. A method according to claim 19, wherein the cryogen is a liquefied gas.

21. A method according to claim 20, wherein the liquefied gas is selected from the group consisting of nitrogen, nitrogen oxides, oxygen, carbon dioxide, liquid air and argon.

22. The method of claim 19, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

23. The method of claim 19, wherein said treating comprises removal of unwanted tissue, removal of an internal or external portion of a lung, or removal of a benign or malignant tumor.

24. The method of claim 19, wherein said treating comprises modulating an immune response, stimulating chondrogenesis, or treating and/or preventing radiation pneumonitis.

25. A method according to claim 19, wherein the tissue is contacted with cryogen or is in proximity to the isotherm for a period of time sufficient to freeze the tissue.

26. A method of claim 19, wherein said therapeutic agent is selected from for the group consisting of oxygen, anti-fungal agents, anti-viral agents, including anti-retroviral agents, anti-microbial agents, anti-rheumatic agents, immunomodulatory agents, steroids or other anti-inflammatory agents, cytokine inhibitors, vasoconstrictors, mucolytics, chemotherapeutic agents, biological response modifiers, vascularization inhibitors, hormone receptor blockers, genes and gene delivery vehicles.

27. A method for treating a tissue in a subject in need thereof, comprising:
identifying the tissue of the subject; and
contacting the tissue with a non-cryogenic gas.

28. The method of claim 27, wherein said non-cryogenic gas is selected from the group consisting of nitrogen, nitrogen oxides, oxygen, carbon dioxide, room air and argon.

29. The method of claim 27, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

30. The method of claim 27, wherein said treating comprises removal of unwanted tissue, removal of an internal or external portion of a lung, or removal of a benign or malignant tumor.

31. The method of claim 27, wherein said treating comprises modulating an immune response, stimulating chondrogenesis, or treating and/or preventing radiation pneumonitis.

32. A method for treating a tissue in a subject in need thereof, comprising:
identifying the tissue of the subject; and
contacting the tissue with a non-cryogenic gas, and
administering a therapeutic agent.

33. The method of claim 32, wherein said non-cryogenic gas is selected from the group consisting of nitrogen, nitrogen oxides, oxygen, carbon dioxide, room air and argon.

34. The method of claim 32, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

35. The method of claim 32, wherein said treating comprises removal of unwanted tissue, removal of an internal or external portion of a lung, or removal of a benign or malignant tumor.

36. The method of claim 32, wherein said treating comprises modulating an immune response, stimulating chondrogenesis, or treating and/or preventing radiation pneumonitis.

37. A method of claim 32, wherein said therapeutic agent is selected from for the group consisting of oxygen, anti-fungal agents, anti-viral agents, including anti-retroviral agents, anti-microbial agents, anti-rheumatic agents, immunomodulatory agents, steroids or other anti-inflammatory agents, cytokine inhibitors, vasoconstrictors, mucolytics, chemotherapeutic agents, biological response modifiers, vascularization inhibitors, hormone receptor blockers, genes and gene delivery vehicles.

38. A method of diagnosing a tissue in a subject in need thereof, comprising:
identifying the tissue of the subject; and
contacting the tissue with a cryogen, or using the cryogen to create an isotherm in proximity to the tissue, and
administering a diagnostic agent.

39. A method according to claim 38, wherein the cryogen is a liquefied gas.

40. A method according to claim 38, wherein the liquefied gas is selected from the group consisting of nitrogen, nitrogen oxides, oxygen, carbon dioxide, liquid air and argon.

41. The method of claim 38, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

42. A method according to claim 38, wherein the tissue is contacted with cryogen or is in proximity to the isotherm for a period of time sufficient to freeze the tissue.

43. A method of claim 38, wherein said diagnostic agents can be selected from radiolabeled substances, haptens, priming agents, imaging agents, fluorescent agents, magnetic marker materials, contrast agents such as X-ray, ultrasound and MRI contrast enhancing agent.

44. A method of diagnosing a tissue in a subject in need thereof, comprising:
identifying the tissue of the subject; and
contacting the tissue with a non-cryogenic gas, and
administering a diagnostic agent.

45. A method according to claim 44, wherein said non-cryogenic gas is selected from the group consisting of nitrogen, nitrogen oxides, oxygen, carbon dioxide, room air and argon.

46. The method of claim 44, wherein said target tissue is selected from the group consisting of an internal or external portion of a lung tissue, pleural tissue and/or chest wall tissue, a lesion, an infected tissue, damaged cartilage, chronically inflamed or diminished cartilage, inflamed tissue, bronchiectatic tissue, asthmatic tissue, exuberant tissue, a stricture in an airway, or tissues afflicted with a benign or malignant tumor, occupational lung disease, pulmonary vascular disease, drug induced lung disease, and acute respiratory distress syndrome.

47. A method of claim 44, wherein said diagnostic agents can be selected from radiolabeled substances, haptens, priming agents, imaging agents, fluorescent agents, magnetic marker materials, contrast agents such as X-ray, ultrasound and MRI contrast enhancing agent.

48. A method according to claims 19, 32, 38 or 44, wherein said agent is delivered prior to contacting the tissue with said cryogen or non-cryogenic gas.

49. A method according to claims 19, 32, 38 or 44, wherein said agent is delivered at the same time as contacting the tissue with said cryogen or non-cryogenic gas.

50. A method according to claims 19, 32, 38 or 44, wherein said agent is delivered after contacting the tissue with said cryogen or non-cryogenic gas.

51. A method according to claims 19, 32, 38 or 44, wherein said agent is mixed with said cryogen or non-cryogenic gas.
